SEQUENCE LISTING (36 sequences)

SEQ ID NO: 1 - DNA, 20 bp, Artificial Sequence
  BACE1 forward primer
gtcggggcagggctactacg

SEQ ID NO: 2 - DNA, 28 bp, Artificial Sequence
  BACE2 reverse primer
ctccaatgatcatgctccctccgacaga

SEQ ID NO: 3 - DNA, 23 bp, Artificial Sequence
  GAPDH forward primer
tttgtcaagctcatttcctggta

SEQ ID NO: 4 - DNA, 24 bp, Artificial Sequence
  GAPDH reverse primer
ttcaaggggtctacatggcaactg

SEQ ID NO: 5 - DNA, 31 bp, Artificial Sequence
  Primer sequence resulting in K->A mutation
ccgggacctccgggcaggtgtgtatgtgccc

SEQ ID NO: 6 - DNA, 31 bp, Artificial Sequence
  Primer sequence resulting in K->Q mutation
ccgggacctccggcagggtgtgtatgtgccc

SEQ ID NO: 7 - DNA, 31 bp, Artificial Sequence
  Primer sequence resulting in K->R mutation
ccgggacctccggaggggtgtgtatgtgccc

SEQ ID NO: 8 - DNA, 39 bp, Artificial Sequence
  Primer sequence resulting in K->A mutation
atcaatggacaggatctggcaatggactgcaaggagtac

SEQ ID NO: 9 - DNA, 39 bp, Artificial Sequence
  Primer sequence resulting in K->Q mutation
atcaatggacaggatctgcaaatggactgcaaggagtac

SEQ ID NO: 10 - DNA, 39 bp, Artificial Sequence
  Primer sequence resulting in K->R mutation
atcaatggacaggatctgagaatggactgcaaggagtac

SEQ ID NO: 11 - DNA, 45 bp, Artificial Sequence
  Primer sequence resulting in K->A mutation
caggatctggcaatggactgcgcagagtacaactatgacaagagc

SEQ ID NO: 12 - DNA, 45 bp, Artificial Sequence
  Primer sequence resulting in K->Q mutation
caggatctgcaaatggactgccaggagtacaactatgacaagagc

SEQ ID NO: 13 - DNA, 45 bp, Artificial Sequence
  Primer sequence resulting in K->R mutation
caggatctgagaatggactgcagggagtacaactatgacaagagc

SEQ ID NO: 14 - DNA, 41 bp, Artificial Sequence
  Primer sequence resulting in K->A mutation
gcgcagagtacaactatgacgcaagcattgtggacagtggc

SEQ ID NO: 15 - DNA, 41 bp, Artificial Sequence
  Primer sequence resulting in K->Q mutation
gccaggagtacaactatgaccagagcattgtggacagtggc

SEQ ID NO: 16 - DNA, 41 bp, Artificial Sequence
  Primer sequence resulting in K->R mutation
gcagggagtacaactatgacaggagcattgtggacagtggc

SEQ ID NO: 17 - DNA, 43 bp, Artificial Sequence
  Primer sequence resulting in K->A mutation
caccaaccttcgtttgcccgcaaaagtgtttgaagctgcagtc

SEQ ID NO: 18 - DNA, 43 bp, Artificial Sequence
  Primer sequence resulting in K->Q mutation
caccaaccttcgtttgccccagaaagtgtttgaagctgcagtc

SEQ ID NO: 19 - DNA, 43 bp, Artificial Sequence
  Primer sequence resulting in K->R mutation
caccaaccttcgtttgcccaggaaagtgtttgaagctgcagtc

SEQ ID NO: 20 - DNA, 35 bp, Artificial Sequence
  Primer sequence resulting in K->A mutation
ccttcgtttgcccgcagcagtgtttgaagctgcag

SEQ ID NO: 21 - DNA, 35 bp, Artificial Sequence
  Primer sequence resulting in K->Q mutation
ccttcgtttgccccagcaagtgtttgaagctgcag

SEQ ID NO: 22 - DNA, 35 bp, Artificial Sequence
  Primer sequence resulting in K->R mutation
ccttcgtttgcccaggagagtgtttgaagctgcag

SEQ ID NO: 23 - DNA, 35 bp, Artificial Sequence
  Primer sequence resulting in K->A mutation
tgtttgaagctgcagtcgcatccatcaaggcagcc

SEQ ID NO: 24 - DNA, 35 bp, Artificial Sequence
  Primer sequence resulting in K->Q mutation
tgtttgaagctgcagtccaatccatcaaggcagcc

SEQ ID NO: 25 - DNA, 35 bp, Artificial Sequence
  Primer sequence resulting in K->R mutation
tgtttgaagctgcagtcagatccatcaaggcagcc

SEQ ID NO: 26 - protein, 11 aa, Homo sapiens
  MOD_RES (1)..(1): ACETYLATION acetylated Lys
KGVYVPYTQGK

SEQ ID NO: 27 - protein, 25 aa, Homo sapiens
  MOD_RES (15)..(15): ACETYLATION acetylated Lys
YYQRQLSSTYRDLRKGVYVPYTQGK

SEQ ID NO: 28 - protein, 20 aa, Homo sapiens
  MOD_RES (20)..(20): ACETYLATION acetylated Lys
REWYYEVIIVRVEINGQDLK

SEQ ID NO: 29 - protein, 13 aa, Homo sapiens
  MOD_RES (9)..(9): ACETYLATION acetylated Lys
VEINGQDLKMDCK

SEQ ID NO: 30 - protein, 7 aa, Homo sapiens
  MOD_RES (7)..(7): ACETYLATION acetylated Lys
EYNYTDK

SEQ ID NO: 31 - protein, 7 aa, Homo sapiens
  MOD_RES (7)..(7): ACETYLATION acetylated Lys
VFEAAVK

SEQ ID NO: 32 - protein, 14 aa, Homo sapiens
  MOD_RES (14)..(14): ACETYLATION acetylated Lys
SIVDSGTTNLRLPK

SEQ ID NO: 33 - protein, 25 aa, Homo sapiens
  MOD_RES (14)..(14): ACETYLATION acetylated Lys
SIVDSGTTNLRLPKKVFEAAVKSIK

SEQ ID NO: 34 - protein, 18 aa, Homo sapiens
  MOD_RES (1)..(1): ACETYLATION acetylated Lys
KVFEAAVKSIKAASSTEK

SEQ ID NO: 35 - protein, 11 aa, Homo sapiens
  MOD_RES (1)..(1): ACETYLATION acetylated Lys
KVFEAAVKSIK

SEQ ID NO: 36 - protein, 11 aa, Homo sapiens
  MOD_RES (8)..(8): ACETYLATION acetylated Lys
KVFEAAVKSIK

(19) United States
(12) Patent Application Publication
(10) Pub. No.: US 2009/0042993 A1
(43) Pub. Date: Feb. 12, 2009

(54) IDENTIFICATION OF NOVEL POST-TRANSLATIONAL PROTEIN MODIFICATIONS

(76) Inventor: Luigi Puglielli, Madison, WI (US)

Correspondence Address: WARF/BHGL, P.O. Box 10395, Chicago, IL, 60610, US

(21) Appl. No.: 12/099,378

(22) Filed: Apr. 8, 2008

Related U.S. Application Data
(60) Provisional application No. 60/923,133, filed on Apr. 11, 2007.

(51) Int. Cl. A61K 31/164; C12Q 1/37; A61P 25/00; A01K 67/027

(52) U.S. Cl. 514/625; 435/23; 800/3

(57) ABSTRACT

Compositions and methods for post-translational modifications that include protein acetylation in the ER lumen and deacetylation in the Golgi apparatus are provided. The disclosed methods are especially suited for the identification of compounds useful for the prevention or treatment of neurodegenerative diseases such as Alzheimer's.

### CROSS-REFERENCE TO RELATED APPLICATIONS

This invention claims priority to U.S. Provisional Patent Application Ser. No. 60/923,133, filed Apr. 11, 2007, which is herein incorporated by reference.

### GOVERNMENT INTERESTS

This invention was made with United States government support awarded by the National Institutes of Health, grant NS 045669. The United States has certain rights in this invention.

### FIELD OF THE INVENTION

The present invention relates generally to post-translational modifications and neurological diseases. More particularly, the present invention relates to BACE1 biochemistry, AT-1 biochemistry, and Alzheimer's disease.

### BACKGROUND

Even though the native conformation of a protein lies encoded in its primary amino acid sequence, the efficiency of folding itself is greatly enhanced by the endoplasmic reticulum (ER) through different complex systems that require chaperones that protect nascent proteins and enzymes that modify, either temporally or definitively, the protein. One system for protein post-translational folding includes BiP and calnexin, whereas another includes oligosaccharyltransferase and UDP-glucose:glycoprotein qlucosyltransferase, which re-attaches one glucose residue to the improperly folded nascent glycoprotein and regulates its interaction with the lectin chaperone calnexin. Once folding is completed, the nascent protein dissociates from the calnexin/calreticulin cycle and moves ahead in the secretory pathway. In contrast, proteins that have not successfully reached an appropriate folding status are removed through ER-associated degradation (ERAD). Therefore, the half-life of a membrane protein is highly affected by the ability to fold correctly and leave the ER in a successful way. The lack of appropriate post-translational modification can make the protein fail quality control and be directed toward ERAD for final degradation.

Reversible acetylation of lysine residues was originally identified in histones and then extended to transcription factors. In recent years a growing number of cytosolic and nuclear proteins have been reported as targets of lysine acetylation, although the specific purpose of lysine acetylation is largely unknown. Recognized functions of lysine acetylation include regulation of activity and molecular stabilization of a protein. From a biochemical point of view, acetylation of a protein requires three components: (i) an appropriate acceptor, i.e., a given protein with the appropriate lysine residue(s); (ii) a donor of the acetyl group -acetyl-CoA; and (iii) an enzyme able to transfer the acetyl group from the donor to the acceptor, which is an acetyl-CoA:lysine acetyltransferase (also simply called acetyltransferase herein). The above components have so far been identified only in the cytoplasm and lysine acetylation has been regarded solely as a cytoplasmic event.

Beta-amyloid (also known as β-amyloid, amyloid β, or Aβ) is a protein fragment suspected of disrupting cell-to-cell communication and damaging cells in Alzheimer's disease (also simply known as Alzheimer's or AD). Beta-amyloid is clipped in a two-stage process from a molecule called amyloid precursor protein (APP). The first cut is made by a protein called β-site APP cleaving enzyme 1, or BACE1, also called β-secretase or memapsin-2. Thus, BACE1 is a key protein in the pathogenesis of Alzheimer's disease in that it serves as the main beta-secretase of the central nervous system. Increases in BACE1 protein or activity are reported in the brain of Alzheimer's disease subjects even in the absence of changes in transcript levels. Control of BACE1 protein levels and activity thus appear to occur primarily at the translational and post-translational levels.

### BRIEF SUMMARY

Methods are provided, which include reacting proteins, ER-derived vesicles, and acetyl-CoA, and measuring the amount of post-translationally modified proteins, where the post-translational modifications comprise acetylation of the proteins in the ER-derived vesicles. In one embodiment of the methods, the proteins are aspartic peptidases. The aspartic peptidases may be BACE1. The ER-derived vesicles may include acetyl-CoA-transporters. For example, the acetyl-CoA-transporters may be AT-1. In the practice of the methods, the proteins may be purified, isolated, or recombinant. The acetylation may include acetylation of one or more lysine residues of the proteins. In some embodiments, the methods may include reacting proteins, ER-derived vesicles, and acetyl-CoA with Golgi vesicles, and measuring the amount of post-translationally modified proteins, where the post-translational modifications include acetylation of the proteins in the ER-derived vesicles, and deacetylation of the proteins in the Golgi vesicles. The methods may be conducted in animal models or in clinical trials. The methods may be conducted in cell cultures.

Methods are provided for treating Alzheimer's disease, which include reducing the acetyl-CoA transport activity of AT-1 in the endoplasmic reticulum of subjects with Alzheimer's disease. The methods may include administering therapeutically effective amounts of at least one inhibitor of AT-1 activity to the subjects, where the inhibitors of AT-1 activity reduce or eliminate the acetyl-CoA transport activity of AT-1 in the endoplasmic reticulum.

Methods are provided for treating Alzheimer's disease, which include reducing the translocation of aspartic peptidases from the ER into the Golgi of subjects with Alzheimer's disease. In the practice of the methods, the aspartic peptidases may be BACE1. The methods may be conducted in animal models or in clinical trials.

In vitro methods are provided for the identification of candidate compounds as compounds that may be useful for the treatment of Alzheimer's disease, where the methods include the steps of: a) providing cells expressing enzymes that acetylate aspartic peptidases in the ER, where the acetylation is required for translocation of the aspartic peptidases into the Golgi; b) contacting the cells with the candidate compounds; and c) measuring the amount of aspartic peptidases translocated from the ER into the Golgi, where a decrease in the amount of aspartic peptidases, relative to the amount of aspartic peptidases translocated from the ER into the Golgi by cells expressing the enzymes that acetylate aspartic peptidases but not contacted with the candidate compounds, identifies the candidate compounds as compounds that may be useful for the treatment of Alzheimer's disease. In one embodiment of the methods, the aspartic peptidase is BACE1. In one embodiment of the methods, the enzyme is AT-1.

In vitro methods are provided for the identification of candidate compounds as compounds that may be useful for the treatment of Alzheimer's disease, where the methods include the steps of: a) providing cells expressing enzymes that acetylate BACE1 in the ER; b) contacting the cells with the candidate compounds; and c) measuring translocation of BACE1 from the ER into the Golgi, where a decrease in translocation, relative to the translocation by cells expressing the enzymes but not contacted with the candidate compounds, identifies the candidate compounds as compounds that may be useful for the treatment of Alzheimer's disease. In one embodiment of the methods, the enzyme is AT-1. In one embodiment of the methods, the cells also express enzymes that deacetylate BACE1 in the Golgi.

Screening methods are provided for testing compounds for their abilities to inhibit acetylation of proteins in the ER. The methods include: a) reacting proteins that are acetylated in the ER, AT-1, and acetyl-CoA; and b) measuring the amount of proteins that are acetylated in the ER, where a decrease in amount of acetylated proteins, relative to the amount of proteins acetylated in the absence of the candidate compounds, identifies the candidate compounds as compounds that may be useful for the inhibition of protein acetylation in the ER. In one embodiment of the methods, the protein that is acetylated in the ER is BACE1. The methods may be conducted in animal models or in clinical trials. The methods may be conducted in cell cultures.

### BRIEF DESCRIPTION OF THE DRAWINGS

FIG. 1 shows images (A-D, F-H), and a graph (E) illustrating how BACE1 is transiently acetylated in the lumen of the ER.

FIG. 2 shows images (A) and a graph (B) illustrating how substitution of the lysine residues blocks the ability of BACE1 to be acetylated in vivo and in vitro.

FIG. 3 shows images (A, D-F) and graphs (B, C) illustrating how transient lysine acetylation controls both translocation along the secretory pathway and molecular stability of the nascent BACE1 protein.

FIG. 4 shows images (A, C, D) and a graph (B) illustrating how both the Lys to Ala and Lys to Arg (loss-of-acetylation) substitutions generate an unstable BACE1 protein that is retained in the early secretory pathway and is degraded by a proteasome-independent system.

FIG. 5 shows graphs illustrating how the ER membrane has an acetyl-CoA transport activity.

FIG. 6 shows graphs illustrating how acetyltransferase and deacetylase activities exist in the lumen of ER and Golgi apparatus, respectively.

FIG. 7 is a schematic model of BACE1 transient lysine acetylation/deacetylation.

FIG. 8 illustrates how the acetylated lysine residues are clustered in a disordered region of BACE1.

FIG. 9 shows images illustrating the non-reducing and reducing migration of BACE1.

FIG. 10 shows graphs illustrating how the ER membrane has an acetyl-CoA transport activity.

FIG. 11 shows a schematic representation of AT-1 (A), and images (B, C) illustrating how AT-1, an ER-located acetyl-CoA transporter, migrates with ER markers.

FIG. 12 shows images (A, B) and graphs (C, D) illustrating how AT-1 localizes in the ER and stimulates acetyl-CoA transport across the ER membrane.

FIG. 13 shows graphs illustrating how acetyl-CoA transport across the ER membrane is inhibited by coenzyme A.

FIG. 14 shows an image (A) and a graph (B) illustrating how AT-1 regulates the steady-state levels of BACE1 and the generation of AP.

FIG. 15 shows graphs illustrating how AT-1 is upregulated in AD brains and following ceramide treatment.

### DETAILED DESCRIPTION OF THE PRESENTLY PREFERRED EMBODIMENTS

The practice of the present invention employs, unless otherwise indicated, conventional techniques of protein chemistry and synthesis, molecular biology (including recombinant techniques), microbiology, cell biology, biochemistry, immunology, protein kinetics, and mass spectroscopy, which are within the skill of art. Such techniques are explained fully in the literature, e.g. in Sambrook et al., 2000, Molecular Cloning: A Laboratory Manual, 3rd ed., Cold Spring Harbor Laboratory Press; Current Protocols in Molecular Biology, Volumes 1-3, John Wiley & Sons, Inc.; Kriegler, 1990, Gene Transfer and Expression: A Laboratory Manual, Stockton Press, New York; Dieffenbach et al., 1995, PCR Primer: A Laboratory Manual, Cold Spring Harbor Laboratory Press, each of which is incorporated herein by reference in its entirety.

As used herein, the terms “a”, “an” and “the” may refer to one or more than one of an item.

Generally, the nomenclature and the laboratory procedures in recombinant DNA technology described below are those well known and commonly employed in the art. Standard techniques are used for cloning, DNA and RNA isolation, amplification and purification. Generally, enzymatic reactions involving DNA ligase, DNA polymerase, restriction endonucleases and the like are performed according to the manufacturer's specifications. Procedures employing commercially available assay kits and reagents are typically used according to manufacturer-defined protocols unless otherwise noted.

“Protein translocation” or “translocation”, as used herein, refers to the translocation of proteins across biological membranes (reviewed, e.g., in Wickner and Schekman, 2005, Science 310: 1452-1456). Eukaryotic proteins are synthesized within the endoplasmic reticulum (ER), are delivered from the ER to the Golgi apparatus (also known as Golgi complex) for post-translational processing and sorting, and are transported from the Golgi to specific intracellular and extracellular destinations. Translocation thus includes transport of proteins from the ER to the Golgi apparatus.

The terms “ER vesicles” and “ER-derived vesicles” are used interchangeably herein. As well, the terms “Golgi vesicles” and “Golgi-derived vesicles” are used interchangeably herein.

“Post-translational modification” refers to the structural modifications of proteins after they have been translated. It is one of the later steps in protein biosynthesis for many proteins. The modifications can be in the form of covalent modifications or non-covalent modifications. Examples of covalent post-translational modifications include signal peptide cleavage, phosphorylation, acetylation, adenylation, proteolysis, amino peptidase clipping, arginylation, disulphide bond formation and cleavage, amidation, glycosylation, isoprenylation, myristoylation, ubiquitination, SUMOlation, and covalent addition of proteins including Agp12 and Nedd8. Examples of non-covalent post-translational modifications include changes in the structure or folding state of a given protein (e.g., denaturation) or its non-covalent interactions with other proteins, nucleic acid, carbohydrate, drugs, compounds or lipids. Some modifications, like phosphorylation, are part of common mechanisms for controlling the behavior of a protein, for instance activating or inactivating an enzyme. Other examples include the binding of a protein as a homo- or hetero-polymer, insertion into a lipid membrane, binding to a glycosyl group, or binding to a drug or compound.

In vitro translation systems may introduce post-translational modifications into translated proteins. Often, these post-translational modifications may be indicative of post-translational modifications that are observed in vivo. Advantageously, the methods of the invention may be used to identify proteins that receive specific types of post-translational modifications. The methods of the present invention are particularly suited for the identification of proteins that are acetylated in the ER. These proteins may further be deacetylated in the Golgi apparatus.

“Acetylation” (or in IUPAC nomenclature “ethanoylation”) describes a reaction that introduces an acetyl functional group into an organic compound, for example into a protein. Deacetylation is the removal of the acetyl group. Acetylation occurs as one type of post-translational modification of proteins, for example, histones and tubulins. Acetylation may occur on particular amino acid residues. An example of that is the reversible acetylation of lysine residues in histones and transcription factors.

The present invention provides applications such as assays for enzymatic activity, and assays for substrate activity. In such assays, the post-translational modifications of proteins may be measured in a variety of ways known in the art. The proteins may be immobilized to some kind of solid phase prior to conducting of the assays. In other applications, it may be desirable to contact the proteins with one or more modifying reagents to introduce post-translational modifications prior to conducting an assay. As well, in other applications, it may be desirable to contact the proteins with a candidate test compound (e.g., candidate inhibitor, agonist, or promoter), prior to conducting an assay, or during conducting the assay. Some proteins may only show an activity after post-translational modification. Thus, an assay of the invention for a characteristic of a protein (for example, binding activity, substrate activity, enzymatic activity, etc.) may comprise the step of treating the protein with a modifying activity to uncover a nascent activity of the protein. Especially preferred treatments are acetylation and deacetylation. This treatment is most preferably applied after immobilization of the protein on a solid phase. This technique enables the screening of proteins in different modification states and analysis of the effect of modifications on activity. In one example, such screening assays may be conducted using cell cultures. Additionally, or in the alternative, such screening assays may be conducted in laboratory animals (animal models), or in clinical trials.

The invention relates, in part, to the production and analysis of proteins formed by the in vitro transcription and translation of nucleic acid constructs. These nucleic acid constructs are characterized by their ability to direct an RNA polymerase to produce an RNA transcript that, in turn, is able to direct the synthesis of a protein or polypeptide sequence. The nucleic acid construct may comprise DNA and/or RNA (preferably, DNA) and may include regions that are single stranded, double stranded (through base pair binding to its complementary sequence), or partially double stranded. The nucleic acid constructs may contain an RNA polymerase promoter sequence that directs the activity of a RNA polymerase to copy at least a portion of the nucleic acid construct to produce an RNA transcript. This transcript, preferably, either encodes a protein or polypeptide or a portion of a protein or polypeptide. The proteins or polypeptides may correspond to amino acid sequences that are found in nature or may be man-made sequences (i.e., sequences not found in nature). The nucleic acid constructs may include synthetic nucleic acid analogs or derivatives that are capable of being copied by transcriptional machinery.

In one aspect, the present invention contemplates the use of protein tags. A “protein tag” is a biochemical indicator. Protein tags, such as affinity tags appended to recombinantly expressed proteins, can serve several purposes, and can be used as a way of purifying proteins using standard conditions rather than developing individual biochemical purifications based on each protein's physical characteristics. For example, a c-Myc-tag can be used for such purposes. The role of tags as an aid to solubilization of a fusion partner has been exploited and maltose binding protein (MBP), glutathione-S-transferase (GST) and thioredoxin have proved useful in this regard. Certain tags can be used as an indicator of fusion protein folding—most notably the Green Fluorescent Protein (GFP). Tags are also useful in providing a common epitope, allowing a single antibody to recognize each fusion protein. Some tags are multifunctional, combining two or more of these roles; for example, the his-tag both permits purification on Ni²⁺-NTA and is used as a common epitope, while GST solubilizes some fusion partners, often increases expression levels, permits purification on glutathione-sepharose and provides a common epitope. The Biotin Carboxyl Carrier Protein (BCCP) tag has found particular utility in array fabrication protocols. This tag is a 79-residue polypeptide derived from the biotin carboxyl carrier domain of the E. coli ACCB protein and is efficiently biotinylated in vivo at a single surface-exposed lysine residue by both prokaryotic and eukaryotic biotin ligases.

The terms “isolated”, “purified”, or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A peptide or protein that is the predominant species present in a preparation is substantially purified. The term “purified” denotes that a peptide or protein gives rise to essentially one band in an electrophoretic gel or HPLC spectrum. Particularly, it means that the peptide or protein is at least 85% pure, more preferably at least 95% pure, and most preferably at least 99% pure.

“Preventing” or “prevention” refers to the preventing or prevention of a disease or medical condition. Examples of a disease or a medical condition are Alzheimer's disease and other neurodegenerative diseases. The compositions and methods of the present invention can be used for prevention of both Alzheimer's and other neurodegenerative diseases. Therefore, as used herein, reference to “prevention of Alzheimer's” is meant to also include the prevention of other neurodegenerative diseases.

“Treating” or “treatment” refers to the treating or treatment of a disease or medical condition. Examples of a disease or a medical condition are Alzheimer's and other neurodegenerative diseases. Therefore, as used herein, reference to “treatment of Alzheimer's” is meant to also include the treatment of other neurodegenerative diseases.

In one aspect, novel methods of post-translational modification (regulation) of proteins are provided. The post-translational regulation of proteins includes acetylation of a particular protein in the lumen of the endoplasmic reticulum (ER), translocation of the acetylated protein from the ER into the Golgi apparatus, and deacetylation of the acetylated protein in the lumen of the Golgi apparatus. The non-acetylated protein intermediates are unable to complete maturation and are enzymatically degraded in the early secretory pathway. In one example, the post-translational modification includes protein acetylation on lysine (abbreviated as Lys or K) residues.

In one example, targets for the prevention and treatment of Alzheimer's disease are provided. The present invention provides methods for identifying proteins whose activity is modulated by this newly identified post-translational mechanism, which proteins could be drug targets for Alzheimer's and other neurodegenerative diseases. In one example, the post-translational regulation of the β-secretase BACE1 can be used for the prevention and treatment of Alzheimer's. In another example, the acetyl-CoA activity of AT-1 may be used as a target for the prevention of Alzheimer's disease. AT-1 regulates the transport of acetyl-CoA.

The present invention teaches that BACE1 is acetylated on seven different lysine residues, all of them facing the N-terminal portion of the nascent protein. The post-translational regulation of BACE1 includes acetylation of BACE1 in the lumen of the endoplasmic reticulum and deacetylation of BACE1 in the lumen of the Golgi apparatus. Specific enzymatic activities acetylate the lysine residues in the ER, and deacetylate the lysine residues in the Golgi apparatus. Dual-enzymatic machinery acts in the ER and Golgi apparatus, respectively, to acetylate and deacetylate the lysine residues. This process regulates one or more of the conformational maturation, translocation along the secretory pathway, and molecular stability of nascent BACE1. The non-acetylated intermediates of BACE1 are unable to complete maturation and are degraded in the secretory pathway. In one aspect, the present invention uses site-directed mutagenesis to show that the transient acetylation of BACE1 is required for the nascent protein to leave the ER and proceed toward the secretory pathway. This process requires carrier-mediated translocation of acetyl-CoA into the lumen of the ER.

Altering the stability of BACE1 alters levels of the beta cleavage of APP to beta-amyloid. Since BACE1 is a significant determinant of Alzheimer's neuropathology, the reduction or elimination of the translocation of BACE1 from the ER into the Golgi can be used for treatment or prevention of Alzheimer's disease.

Furthermore, this process of post-translational modification of BACE1 is stimulated by a lipid second messenger molecule normally present in the brain, called ceramide. Ceramide regulates the rate of P cleavage of the Alzheimer's disease amyloid precursor protein by affecting the molecular stability of the β secretase BACE1. Such an event is stimulated in the brain by the normal process of aging, and is under the control of the general aging programming mediated by the insulin-like growth factor 1 receptor. Thus, BACE1 stability can be altered through modulating levels of ceramide. Increasing the amount of ceramide stabilizes BACE1, which in turn leads to greater accumulation of beta amyloid.

In vivo, the form of post-translational modification described herein occurs downstream of IGF1-R, the common regulator of lifespan, to regulate the rate of amyloid β-peptide generation during aging. Therefore, regulation of this post-translational modification might provide novel targets to prevent the Alzheimer's risk associated with aging.

The methods of the invention provide a facile and specific assay to screen compounds as potential Alzheimer's drugs. In the present invention BACE1 may be a purified or partially purified native protein, a cloned BACE1 or an engineered (recombinantly produced) variant thereof, a crude preparation of the BACE1 protein, an extract containing BACE1 activity, or any mixture of two or more of the above. The protein BACE1 is preferably from a mammalian (e.g., human, non-human animal, etc.) source. Fragments of BACE1 that retain BACE1 activity are also within the scope of the invention.

As well, in the present invention AT-1 may be a purified or partially purified native protein, a cloned AT-1 or an engineered (recombinantly produced) variant thereof, a crude preparation of the AT-1 protein, an extract containing AT-1 activity, or any mixture of two or more of the above. The protein AT-1 is preferably from a mammalian (e.g., human, non-human animal, etc.) source. Fragments of AT-1 that retain AT-1 activity are also within the scope of the invention. Identification and molecular characterization of AT-1 acetyl-CoA transporters are described in, for example, Kanamori et al., 1997, Proc, Natl. Acad. Sci. USA 94: 2897-2902, and in U.S. Pat. No. 5,851,788.

A compound that interacts with AT-1 may be one that is a substrate for AT-1 enzyme, one that binds AT-1, or one that otherwise acts to alter AT-1 activity by binding to an active or an alternate site. The compound that interacts with AT-1 can be labeled to allow easy quantitation of the level of interaction between the compound and AT-1. Examples of useful radiolabels are tritium (³H) or radiocarbon (¹⁴C). In some embodiments of the present invention, compounds that mimic CoA may be used to prevent or block the transport activity of AT-1, and thus may be helpful for the prevention and/or treatment of AD. The transport activity of AT-1 may be down-regulated by down-regulating the amount of AT-1 produced, for example using recombinant antisense approaches.

“Therapeutically effective amount” refers to that amount of a composition, which results in amelioration of symptoms or a prolongation of survival in a subject. A therapeutically relevant effect relieves to some extent one or more symptoms of a disease or condition or returns to normal either partially or completely one or more physiological or biochemical parameters associated with or causative of the disease or condition.

Methods for designing assays for the presence of a known target in Alzheimer's disease are also provided. Examples of such targets for Alzheimer's disease are BACE1 and AT-1. As well, methods for assaying for the identification of modulators of post-translational modification pathways that play a role in Alzheimer's progression are provided. These modulators may be novel drugs for the prevention or treatment of Alzheimer's. Alternatively, the discovery of such modulators may lead to the discovery of new drug targets and/or drug candidates for Alzheimer's. Such modulators may include regulators of the acetyl-CoA transport activity of AT-1. For example, such modulators may include BACE1 ligands. The modulators may also be BACE1 inhibitors. The assays may be conducted in vitro and/or in vivo. Conducted in vitro, such assays may include post-translation modification of proteins using ER-derived vesicles. ER-derived vesicles and Golgi-derived vesicles can be obtained using methods known in the art, e.g. as described in the examples section below, and also as described by Kuehn et al., 1998, Nature 391: 187-190; Costantini et al., 2006, EMBO J. 25: 1997-2006. In vitro, such assays may be conducted in cell cultures. These cells may express BACE1; alternatively, the cells may overexpress BACE1; as well, the cells may underexpress BACE1. In vivo, such assays may be conducted in laboratory animals (animal models), or they may be conducted in clinical trials. For example, wild-type (non-transgenic) and transgenic animals can be treated with compounds that block AT-1 activity; brain homogenates are then tested for BACE1 steady-state levels and APP processing by immunoblotting, whereas the generation of Aβ can be analyzed by sandwich ELISA. Conversely, animal models of AD (transgenic) can be treated with the same compounds and the progression of AD-like neuropathology can be analyzed by conventional histological approaches and/or memory assessment.

Screens for compounds that can be used as inhibitors, ligands, or promoters of the post-translational modifications described herein are also contemplated. These compounds may find use for developing drugs or treatments for Alzheimer's disease. In general, compounds are identified from large libraries of both natural product extracts (for example, obtained from plant, microorganism or animal sources) and synthetic (or semi-synthetic) extracts or chemical libraries according to methods known in the art. The precise source of test extracts or compounds is not critical to the screening procedures of the invention. Accordingly, virtually any number of chemical extracts or compounds can be screened using the methods described herein. Examples of such extracts or compounds include, but are not limited to, fungal-, plant-, prokaryotic-, or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), and Harbor Branch Oceanographic Institute (Ft. Pierce, Fla.). Natural and synthetically produced libraries are produced, if desired, according to methods known in the art, e.g., by extraction and fractionation. If desired, any library or compound is readily modified using standard chemical, physical, or biochemical methods.

When a crude extract is found to decrease activity of the tested enzyme (e.g., AT-1) in the ER, further fractionation of the positive lead extract is necessary to isolate chemical constituents responsible for the observed effect. Thus, the goal of the extraction, fractionation, and purification process is the careful characterization and identification of a chemical entity within the crude extract having the desired activity. Methods of fractionation and purification of such heterogeneous extracts are known in the art. If desired, compounds shown to be useful agents for the treatment of pathogenicity are chemically modified according to methods known in the art.

One embodiment of the present methods is based on test compound-induced inhibition of AT-1 activity. The AT-1 inhibition assay involves adding AT-1 or an extract containing AT-1 to mixtures of AT-1 substrate (e.g., BACE1) and the test compound, both of which are present in known concentrations. The assay is carried out with the test compound at a series of different dilution levels. After a period of incubation, the labeled portion of the substrate released by the AT-1 enzymatic action is separated and counted. The assay is generally carried out in parallel with a control (no test compound) and a positive control (containing a known enzyme inhibitor instead of a test compound).

Methods for assaying for BACE1 inhibitors, ligands, or promoters are also provided. The screens can be conducted to identify compounds such as drugs that can block or diminish BACE1 activity. Such BASE1 inhibitors (e.g., BACE1-inhibiting drugs) can help prevent the build up of beta-amyloid, and thus may help slow or stop the Alzheimer's disease.

In certain embodiments of the invention, translation and, optionally, transcription of nucleic acid constructs to produce proteins (e.g. BACE1, AT-1) is carried out in an assay plate used for carrying out an assay for an activity of interest. For example, plates having assay domains coated with (i) a substrate of an activity, (ii) a binding reagent specific for a substrate of an activity, (iii) a binding reagent specific for a product of an activity, (iv) a binding reagent specific for a post-translational modification of the translation products, (v) a binding reagent suitable for immobilizing the translation products, etc., may be used. The assays of the invention are then carried out in this same plate. Alternatively, the translation reaction is carried out in one container, e.g., the well of a multi-well plate and the translation products transferred to an assay plate for analysis.

The assays of the invention may detect or utilize binding interactions of binding species. “Binding species” is used to describe a molecular species that is able to bind to another molecular species, its binding partner. These binding interactions are characterized in that they are non-covalent, or covalent having dissociation constants which are lower than 1 mM, preferably lower than 100 μM, more preferably lower than 10 μM, and most preferably higher than 1 μM. Examples of binding species and binding partners include biotin, antibodies, streptavidin, avidin, EDTA, chelates (e.g., EDTA, NTA, IDA), antigens, fluorescein, haptens (e.g., fluorescein, digoxigenin), proteins, peptides, drugs, nucleic acids, nucleic acid analogues, lipids, carbohydrates, protein A, protein G, protein L, receptors, ligands, inhibitors, lectins, enzymes, substrates, transition state analogues, mechanism based inhibitors, epitopes, affinity tags (e.g., epitope tags such as his(6), glutathione, Myc, S-tag, T7-Tag), etc.

The assays of the invention may utilize labels. “Label” or “detectable label” is used to describe a substance used to detect (directly or indirectly) a molecular species. The label may be the molecular species itself or it may be linked to the molecular species. In certain embodiments of the invention, labels are used in order to follow or track a given molecular species, for example, to determine its distribution and concentration (as, for example, a radio-labeled drug molecule is used to determine its pharmacological properties when introduced into an animal or human subject). In alternative embodiments, a label is introduced into a binding species so as to allow the binding species to be used to track and/or determine the presence and/or amount of a binding partner of the binding species. For example, immunoassays using labeled antibodies (e.g., antibodies labeled with ECL labels) can be used to detect and determine binding partners (analytes) bound by the antibody. The term “label” also includes indirect labeling of proteins using detectable labels bound to other molecules or complexes of molecules that bind to a protein of interest, including antibodies and proteins to which antisera or monoclonal antibodies specifically bind. As used herein, the term “calorimetric label” includes a label that is detected using an enzyme-linked assay.

In some embodiments of the invention, labels are used which may be detected directly, e.g., on the basis of a physical or chemical property of the label, including but not limited to optical absorbance, fluorescence, phosphorescence, chemiluminescence, electrochemiluminescence, refractive index, light scattering, radioactivity, magnetism, catalytic activity, chemical reactivity, etc. Examples of directly detectable labels include radioactive labels, fluorescent labels, luminescent labels, enzyme labels, chemiluminescent labels, electrochemiluminescent labels, phosphorescent labels, light scattering or adsorbing particles (e.g., metal particles, gold colloids, and silver colloids), magnetic labels, quantum dots, etc. In other embodiments of the invention, labels are used which may be detected indirectly via interactions with species comprising directly detectable labels. The indirectly detectable labels are, preferably, binding species; these binding species readily allow the binding to a binding partner that is labeled with a directly detectable label. Examples of indirectly detectable labels include binding species as described above, for example, antibodies, antigens, haptens, avidin, biotin, streptavidin, fluorescein, nucleic acid sequences, nucleic acid analogue sequences, epitope tags (such as myc, FLAG, GST, MBP, V5), digoxigenin, etc.

The invention relates, in part, to the production of proteins in cell-free systems and the analyses of these proteins. Protein synthesis can be accomplished using cell lysates or extracts (crude or partially purified) that contain the machinery necessary for protein synthesis. This machinery is found in most living cells; however, certain cell types are preferred because of their high protein synthesis activity. Three preferred translation systems for producing proteins are the bacterial (more preferably E. coli extract, most preferably E. coli S30 cell free extract), plant germ (most preferably, wheat germ) and reticulocyte (most preferably, rabbit reticulocyte) lysate translation systems. Optionally, the cell lysates are supplemented with components such as ATP, tRNA, amino acids, RNA polymerases, microsomes, protease inhibitors, proteosome inhibitors, etc., that enhance the functioning of the translation machinery, provide a missing component of the machinery, inhibit protein degradation, or provide an additional activity such as transcription or post-translational modification. In an alternate embodiment of the invention the cell-free system is reconstituted from individual purified or partially purified components, e.g., reconstituted E. coli translation system using recombinant components as described in Shimizu et al., 2001, Nat. Biotech. 19: 751-755.

In another alternate embodiment, a cell-based translation system is used. The E. coli systems are advantageous when a gene has been cloned into a vector with prokaryotic regulatory sequences, such as the promoter and ribosome binding site. These systems are coupled in that transcription and translation can occur simultaneously. The E. coli extract systems have been the subject of much optimization and allow the production of mg amounts of protein using large scale (1 ml) reactions fed with reagents through semi-permeable membranes.

Wheat germ- and the rabbit reticulocyte-based cell free systems are suitable for the translation of mRNA into proteins. These lysates can be supplemented with RNA polymerases so that they carry out both transcription, to generate the mRNA, and translation to produce protein thus producing what is called a coupled system as seen in E. coli. The lysates based on the wheat germ and the rabbit reticulocyte may also be supplemented with other components in order to introduce additional activities to these lysates. In one embodiment, a lysate is supplemented with microsomes (preferably, dog pancreatic microsome, preparations) or Xenopus oocyte extract to allow for certain post-translation modifications as well as the processing of proteins which are secreted, inserted or associated with membranes.

In some embodiments of the invention, solid phase supports are used for purifying, immobilizing, or for carrying out solid phase activity assays for analyzing the activity of one or more expressed proteins. Examples of solid phases suitable for carrying out the methods of the invention include beads, particles, colloids, single surfaces, tubes, multiwell plates, microtitre plates, slides, membranes, gels and electrodes. When the solid phase is a particulate material it is, preferably, distributed in the wells of multi-well plates to allow for parallel processing of the solid phase supports. Proteins of interest or other assay reagents may be immobilized on the solid phase supports, e.g., by non-specific adsorption, covalent attachment or specific capture using an immobilized capture reagent that binds, preferably specifically, the protein or assay reagent of interest. Immobilization may be accomplished using proteins or assay reagents that are labeled with binding species that form binding pairs with immobilized capture reagents. Optionally, a protein is immobilized on a solid phase, then the solid phase is washed and the protein is analyzed. The wash step allows for the rapid purification of the protein from other, potentially interfering, components of the translation reaction. Optionally, a protein is treated, prior to analysis, to add or remove post-translational modifications.

In one embodiment of the invention, a nucleic acid construct is obtained with the gene (or genes) of interest located within the construct such that the gene(s) of interest can be transcribed into RNA that can direct the synthesis of the protein encoded by the genes of interest. The nucleic acid construct is then subjected to an in vitro transcription reaction and followed by an in vitro translation reaction. These two reactions can also be combined in a single in vitro reaction. The protein product (or, alternatively, protein obtained by any other method, e.g. purified) is then analyzed for the presence of post-translational modifications of interest. During the translation reaction the protein produced in the transcription and translation reaction can also be labeled using a modified tRNA that directs the incorporation of a modified amino acid during this step. Preferably, a plurality of constructs comprising different genes are prepared and the protein products of these genes produced and analyzed, most preferably in parallel in the wells of multi-well plates.

Following the transcription and translation reaction the proteins produced in the mix may be captured onto a solid phase. Examples of solid phases suitable for carrying out the methods of the invention include beads, particles, colloids, single surfaces, tubes, multiwell plates, microtitre plates, slides, membranes, gels and electrodes. The capture of the proteins is achieved using a binding partner specific for the modification introduced by the modified tRNA during the translation reaction. A good example of how this is achieved is by the use of the biotin-lys-tRNA that results in a protein containing lysine residues modified with biotin. The biotinylated proteins produced in this way are then captured onto a solid phase using an avidin or streptavidin coated solid phase. Alternative methods for capture of the proteins produced include non-specific or passive adsorption, and via binding to specific binding partners other than those for the modified amino acids introduced by the addition of modified tRNA (e.g., binding partners of affinity tags present in the expressed protein). Alternatively the proteins produced may be generated without the use of the modified tRNA and captured using alternative methods for capture, including non-specific or passive adsorption, and via the binding to specific binding partners for the produced proteins (e.g., binding partners of affinity tags present in the expressed proteins).

In certain embodiments of the invention, a protein expressed by the expression methods of the invention is characterized by its catalytic activity or its ability to act as a substrate in the presence of a catalytic activity or other chemical activity. The activity may be measured by using a substrate that exhibits a detectable change in a chemical or physical property when acted on by the activity of interest (e.g., luminescence, color, ability to act as an ELC coreactant, mobility shift on a gel, mass spectrometry, etc.).

The post-translational modifications are carried out by compounds located in ER vesicles, Golgi vesicles, or combinations of ER vesicles and Golgi vesicles, and this is readily detected using antibodies specific to acetylation. In one example an antibody is labeled with a detectable label and the bound signal detected to determine the amount of acetylation for a given protein. Alternatively, the post- post-translational modifications of the present invention can be detected using mass spectrometry using methods known in the art.

In a further embodiment of the invention a labeled substrate for a given post-translation modification is included in the translation reaction. An example of such substrate includes BACE1. Examples of labels that could be used include, metal chelates, biotin, binding species, digoxigenin, enzymes, fluorophores, luminescent species and ECL labels. The result of the inclusion of a labeled substrate for a given post-translational reaction is that those proteins that are subjected to this post-translational modification are produced with the post-translational modification including the label. This results in the production of a labeled protein. Proteins that are labeled in this way through the action of a post-translational modification and are also labeled as described using modified tRNA contain two, preferably different, labels. Thus these proteins are readily detected by immobilization through one of the labels and detection with the other.

In an alternative embodiment the invention involves either obtaining clones or the production of clones in a desired cloning vector or nucleic acid construct. The DNA from these is then subjected to an in vitro transcription reaction and followed by an in vitro translation reaction. These two reactions can also be combined in a single in vitro reaction. During the translation reaction the protein produced is also labeled using a modified tRNA that directs the incorporation of a modified amino acid during this step. On completion of this protein expression step the reaction mix is then subjected to assays for post-translational modification. The protein in the mix is then captured onto a solid phase using a binding protein specific for the post-translational modification. A good example of how this is achieved is by the use of an antibody specific for a post-translational modification for example an anti-acetylated Lysine antibody immobilized onto a solid phase. This allows the capture of proteins that have been acetylated onto the solid phase. Examples of the solid phase include beads, particles, colloids, single surfaces, tubes, multiwell plates, microtitre plates, slides, membranes, gels and electrodes. With these proteins immobilized onto a solid phase the proteins are then available for an assay to determine if they have been captured and thus subject to any structural modifications or post-translational modifications. These proteins are then detected using a binding species specific for the modified amino acid that has an attached label.

### EXAMPLES

It is to be understood that this invention is not limited to the particular methodology, protocols, subjects, or reagents described, and as such may vary. It is also to be understood that the terminology used herein is for the purpose of describing particular embodiments only, and is not intended to limit the scope of the present invention, which is limited only by the claims. The following examples are offered to illustrate, but not to limit the claimed invention.

Cell Cultures and Cell Treatment

Chinese Hamster Ovary (CHO) cells were grown in DMEM (Gibco BRL—Invitrogen, Carlsbad, Calif.) supplemented with 10% fetal bovine serum (FBS) and penicillin/streptomycin (Mediatech, Inc., Herndon, Va.) as described before (Puglielli et al., 2001, Nat. Cell Biol. 3: 905-912; Puglielli et al., 2003, J. Biol. Chem. 278: 19777-19783; Costantini et al., 2005, Biochem. J. 391: 59-67; Costantini et al., 2006, EMBO J. 25: 1997-2006). Cells were maintained in a humidified atmosphere with 5% CO₂. For ceramide treatment, C6-ceramide (also simply called ceramide herein) was administered at the final concentration of 10 μM; treatment was started when cells were ˜30% confluent and carried out for 4 days, unless specified otherwise. Treatment with proteasome inhibitors MG-132 (5 μM) and lactacystin (10 μM) was done for 10 hours, as described earlier (Qing et al., 2004, FASEB J. 18: 1571-1573).

For neuronal cultures, hippocampi and frontal cortices were dissected from embryonic-day 16-18 (E16-18) mice and placed in DMEM (Gibco BRL, a division of Invitrogen, Carlsbad, Calif.) (Costantini et al., 2006, EMBO J. 25: 1997-2006). The tissue was mechanically dissociated by pipetting and neurons were plated on poly-(L-lysine)-coated 6-well plates (Becton Dickinson Labware) for two hours. Neurons were then changed to Neurobasal medium containing 2% B27 supplement (Gibco BRL) in the absence of serum or antibiotics. Cultures grown in serum-free media yielded ˜99.5% neurons and 0.5% glia. Microscopically, glial cells were not apparent in cultures at the time they were used for experimental analyses. Medium was changed every 3 days.

Cell Extraction and Immunoprecipitation

Cell lysates were prepared in GTIP buffer (100 mM Tris-pH 7.6, 20 mM EDTA, 1.5 M NaCl) with 1% Triton X-100 (Roche, Basel, Switzerland), 0.25% NP40 (Roche), and a protease inhibitor cocktail (Roche) as described before (Puglielli et al., 2003, J. Biol. Chem. 278: 19777-19783; Costantini et al., 2005, Biochem. J. 391: 59-67). Briefly, cell monolayers were washed twice in PBS, scraped, transferred into eppendorf tubes, and centrifuged at 8,000 rpm for 5 min in a table-top centrifuge. Cell pellets were then lysed in ice for 30 min with the buffer indicated above, and centrifuged again at 14,000 rpm prior to the recollection of the protein extracts. Proteins were quantitated with the BCA Protein Assay (Pierce, Rockford, Ill.) following the manufacturer's instructions.

For the immunoprecipitation of BACE1, cell lysates were pre-cleared with BioMag Protein A (Polysciences, Inc., Warrington, Pa.); beads were used at the concentration of 15 μl of the slurry per sample and separated with a magnetic holder. BACE1 was then immunoprecipitated with anti-BACE1 antibodies overnight and recovered with BioMag Protein A as above. The immunoprecipitate-bead complexes were washed 3 times in PBS and boiled for 10 min in SDS sample buffer (Invitrogen) with β-mercaptoethanol. Beads were then separated and the samples were analyzed by reducing SDS-PAGE.

Endo H Digestion

Digestion with Endoglycosidase H (Endo H) was carried out as described by Nohturfft et al., 1999, Proc. Natl. Acad. Sci. USA 96: 11235-11240. Briefly, ER and Golgi vesicles were resuspended in 0.1 ml of buffer A (10 mM Hepes•KOH, pH 7.4, 100 mM NaCl, 10 mM KCl, 1.5 mM MgCl₂, 5 mM sodium EDTA, 5 mM sodium EGTA, 250 mM sucrose), and then denatured in the presence of 2% SDS and 4% 2-mercaptoethanol, and heated at 100° C. for 10 min. For treatment with Endo H, samples received sequential additions of 9 μl of 0.5 M sodium citrate (pH 5.5), 5 μl of solution containing 17× protease inhibitors, followed by 1 μl of Endo H (0.05 units). All reactions were carried out overnight at 37° C. and stopped by addition of 20 μl of buffer B (0.25 M Tris•HCl, pH 6.8, 2% SDS, 10% (vol/vol) glycerol, 0.05% (wt/vol) bromophenol blue, 4% 2-mercaptoethanol). The mixtures were then heated at 100° C. for 5 min and subjected to SDS/PAGE.

Protein Digestion and LC-MS/MS Analysis

BACE1-myc was purified via affinity chromatography on a ProFound c-Myc-Tag IP/Co-IP kit (Pierce). Each BACE1 preparation was then precipitated by addition of 4 volumes of acetone, pelletted by centrifugation, washed, and vacuum-dried. The protein pellets were suspended in 40 μl of 8 M urea, 1 mM DTT, and heated to 37° C. for 5 min prior to dilution in 280 μl of 50 mM ammonium bicarbonate pH 7.5, 1 mM DTT. Proteolysis was then initiated by addition of 0.8 μg of sequencing-grade modified trypsin (Promega, Madison, Wis.) and continued for 8 to 16 hr at 37° C. Digestion was terminated by addition of formic acid to 0.5%, and peptides were purified by solid phase extraction using a C18-ZipTip (Millipore, Billerica, Mass.) for subsequent μLC-MS and μLC-MS/MS analyses.

A portion of each purified digest, corresponding to 10 μg of pre-digested protein, was analyzed by μLC-MS/MS using a Micromass Q-TOF2 spectrometer to locate acetylation sites. Chromatographic separation of peptides prior to mass spectral analysis was accomplished using C18 reverse phase HPLC columns made in-house from which eluted species are directly micro-electrosprayed. Columns were made using lengths of fused silica tubing (365 μm o.d., 100 μm i.d.) with pulled tips (1 μm orifice) that were packed to 12 cm with Zorbax Eclipse XDB-C18, 5 μm, 300 Å pore size media. An Agilent 1100 series HPLC (Palo Alto, Calif.) delivered solvents A: 0.1% (vol/vol) formic acid in water, and B: 95% (vol/vol) acetonitrile, 0.1% (vol/vol) formic acid at either 1 μl/min for sample application, or 150-200 nl/min during a 200 min 2% (vol/vol) B to 70% (vol/vol) B gradient. Voltage was applied upstream of the column, by introduction of a platinum wire electrode into the fluid path via a PEEK T-junction. As peptides eluted from the HPLC-column/electrospray source, MS/MS spectra were collected; redundancy was limited by dynamic exclusion. Charge dependent collision energy profiles were empirically pre-determined. MS/MS data were converted to pkl file format using MassLynx version 3.5 (Micromass Waters, Milford, Mass.). Resulting pkl files were used to search the full International Protein Index (IPI) database using the Mascot (Matrix Science, London, UK) search engine. Putative acetylated peptides identified by Mascot were confirmed and sequences were assigned to MS/MS spectra manually. The remaining 10 μg of each digest was analyzed by μLC-MS (same gradient and conditions as above) to allow integration of uninterrupted ion current for the direct comparison of acetylated and non-acetylated peptide signals.

Mutagenesis and mRNA Quantitation

Mutagenesis of BACE1 was performed with QuikChange® Multi Site-Directed Mutagenesis Kit (Stratagene, La Jolla, Calif.) according to the manufacturer's protocol. The primers used are listed in Table 1. The presence of the mutations was confirmed by sequencing (University of Wisconsin Biotechnology Center, DNA Sequencing Facility). BACE1 was then excised with EcoRI digestion and inserted in pcDNA3.1/Zeo (Invitrogen). The correct orientation of the insert was verified by sequencing. Transfection of CHO cells was carried out by using Lipofectamine 2000 (Invitrogen) as described before (Puglielli et al., 2001, Nat. Cell Biol. 3: 905-912; Puglielli et al., 2003, J. Biol. Chem. 278: 19777-19783; Costantini et al., 2005, Biochem. J. 391: 59-67; Costantini et al., 2006, EMBO J. 25: 1997-2006).

For mRNA quantitation of BACE1_{WT}, BACE1_{Ala, and BACE}1_{Gln} expressing cells, total RNA was extracted and isolated by using the RNeasy Mini kit (Qiagen, Valencia, Calif.) according to manufacturer's protocol. RT-PCR was performed with Illustra Ready-To-Go RT-PCR Beads (Amersham, Piscataway, N.J.) according to manufacturer's protocol. Primers for BACE1: forward 5′-GTCGGGGCAGGGCTACTACG-3′ (SEQ ID NO:1); and reverse 5′-CTCCMTGATCATGCTCCCTCCGACAGA-3′ (SEQ ID NO:2); and primers for GAPDH: forward 5′-TTTGTCAAGCTCATTTCCTGGTA-3′ (SEQ ID NO:3); and reverse 5′-TTCMGGGGTCTACATGGCAACTG-3′ (SEQ ID NO:4), were designed inside the ORF and produced fragments of the expected size. The list of primers used for BACE1 mutagenesis is shown in Table 1. The nucleotides triplets listed in bold indicate the positions of the mutations.

TABLE 1
List of primers used for BACE1 mutagenesis

| | | |
|---|---|---|
| Position | Mutation | Primer sequences |

| | | | |
|---|---|---|---|
| 126-1^{st} | K → A | CCGGGACCTCCGGGCAGGTGTGTATGTGCCC | (SEQ ID NO:5) |
| | K → Q | CCGGGACCTCCGGCAGGGTGTGTATGTGCCC | (SEQ ID NO:6) |
| | K → R | CCGGGACCTCCGGAGGGGTGTGTATGTGCCC | (SEQ ID NO:7) |
| 275-1^{st} | K → A | ATCAATGGACAGGATCTGGCAATGGACTGCAAGGAGTAC | (SEQ ID NO:8) |
| | K → Q | ATCAATGGACAGGATCTGCAAATGGACTGCAAGGAGTAC | (SEQ ID NO:9) |
| | K → R | ATCAATGGACAGGATCTGAGAATGGACTGCAAGGAGTAC | (SEQ ID NO:10) |
| 279-2^{nd} | K → A | CAGGATCTGGCAATGGACTGCGCAGAGTACAACTATGACAAGAGC | (SEQ ID NO:11) |
| | K → Q | CAGGATCTGCAAATGGACTGCCAGGAGTACAACTATGACAAGAGC | (SEQ ID NO:12) |
| | K → R | CAGGATCTGAGAATGGACTGCAGGGAGTACAACTATGACAAGAGC | (SEQ ID NO:13) |
| 285-3^{rd} | K → A | GCGCAGAGTACAACTATGACGCAAGCATTGTGGACAGTGGC | (SEQ ID NO:14) |
| | K → Q | GCCAGGAGTACAACTATGACCAGAGCATTGTGGACAGTGGC | (SEQ ID NO:15) |
| | K → R | GCAGGGAGTACAACTATGACAGGAGCATTGTGGACAGTGGC | (SEQ ID NO:16) |
| 299-1^{st} | K → A | CACCAACCTTCGTTTGCCCGCAAAAGTGTTTGAAGCTGCAGTC | (SEQ ID NO:17) |
| | K → Q | CACCAACCTTCGTTTGCCCCAGAAAGTGTTTGAAGCTGCAGTC | (SEQ ID NO:18) |
| | K → R | CACCAACCTTCGTTTGCCCAGGAAAGTGTTTGAAGCTGCAGTC | (SEQ ID NO:19) |
| 300-2^{nd} | K → A | CCTTCGTTTGCCCGCAGCAGTGTTTGAAGCTGCAG | (SEQ ID NO:20) |
| | K → Q | CCTTCGTTTGCCCCAGCAAGTGTTTGAAGCTGCAG | (SEQ ID NO:21) |
| | K → R | CCTTCGTTTGCCCAGGAGAGTGTTTGAAGCTGCAG | (SEQ ID NO:22) |
| 307-3^{rd} | K → A | TGTTTGAAGCTGCAGTCGCATCCATCAAGGCAGCC | (SEQ ID NO:23) |
| | K → Q | TGTTTGAAGCTGCAGTCCAATCCATCAAGGCAGCC | (SEQ ID NO:24) |
| | K → R | TGTTTGAAGCTGCAGTCAGATCCATCAAGGCAGCC | (SEQ ID NO:25) |

Disorder Prediction Analysis

Prediction was mainly done by using the DRIP-PRED analysis system of the Stockholm Bioinformatics Center, Stockholm University, Sweden. However, the results were further analyzed by using additional prediction programs, which included GLOBPLOT 2 and DisEMBL (Linding et al., 2003, Nucleic Acids Res. 31: 3701-3708; Linding et al., 2003, Structure 11: 1453-1459). For the prediction the target sequence is processed to produce a PSI-BLAST profile (Altschul et al., 1997, Nucleic Acids Res. 25: 3389-3402; Jones, 1999, J. Mol. Biol. 292: 195-202; MacCallum, 2004, Bioinformatics 20: 1224-1231). Every window of 15 residues centered on a residue is mapped to a node on the UniProt SOM, and baseline disorder prediction score for this residue is taken from the corresponding position in the log matrix. The score is distributed around zero, with positive values indicating disorder. A confident secondary structure prediction suggests that there is some ordered structure (Jones, 1999, J. Mol. Biol. 292: 195-202; Jones and Ward, 2003, Proteins 53: 573-578), so the score is set to −0.5 when the numerical PSIPRED outputs (H for helix, E for strand, C for coil) for that residue satisfy the following: H−(E+C)>0.5 or E−(H+C)>0.5. The scores are then smoothed with four cycles of a ±residue window average, and then adjusted by +0.5, and finally capped to the range 0-1.

Antibodies and Western Blot Analysis

Western blotting was performed on 10% Bis-Tris, 4-12% Bis-Tris, or 7% Tris-Acetate SDS-PAGE systems (NuPAGE; Invitrogen) as described (Puglielli et al., 2001, Nat. Cell Biol. 3: 905-912; Puglielli et al., 2003, J. Biol. Chem. 278: 19777-19783; Costantini et al., 2005, Biochem. J. 391: 59-67; Costantini et al., 2006, EMBO J. 25: 1997-2006). The following antibodies were used: anti-BACE1 N-terminal (monoclonal antibody; R&D Systems, Minneapolis, Minn.); anti-BACE1 C-terminal (polyclonal antibody; Abcam, Cambridge, Mass.); anti-pro-BACE1 (polyclonal; Abcam); anti-acetylated lysine (monoclonal; Abcam); anti-calreticulin (ER marker, polyclonal; Abcam); anti-58 K Golgi/formiminotransferase cyclodeaminase (Golgi marker, polyclonal; Abcam); anti-syntaxin (Golgi marker, monoclonal; Abcam); anti-EEA1 (endosomes, monoclonal; BD Transduction Laboratories, Lexington, Ky.); anti-actin (polyclonal; Cell Signaling, Danvers, Mass.). Secondary antibodies (Amersham) were used at a 1:6000 dilution. Binding was detected by chemiluminescence (LumiGLO kit; KPL, Gaithersburg, Md.). Only a representative blot of at least three different experiments is shown throughout the paper.

Pixel densities (for signal-area) of scanned images were calculated with Adobe Photoshop (Adobe Systems, Inc., San Jose, Calif.); densitometry (for signal-density) was analyzed with the EpiChemi3 Darkroom (UVP Bioimaging Systems, Upland, Calif.) using Labworks Image Acquisition and Analysis Software 4.5 (UVP).

In Vitro Acetylation

BACE1-myc was purified from stably-transfected CHO cells with the ProFound c-Myc-Tag IP/Co-IP Kit (Pierce). BACE1-myc was incubated with the purified recombinant HAT fragment of p300 (5U; Upstate Biotechnology—Millipore, Billerica, Mass.) in the presence of [³H]Acetyl-CoA (1000 cpm/μmol) (200 mCi/mmol; American Radiolabeled Chemicals) for 1 hr at 30° C. The reaction was performed in acetylation buffer (50 mM Tris HCl (pH 8.0), 0.1 mM EDTA, 1 mM DTT, 10% glycerol, 20 μM acetyl-CoA) and stopped by lowering the temperature to 0-4° C.

BACE1 was immunoprecipitated with an anti BACE1 N-terminal monoclonal antibody and then counted on a liquid scintillation counter. As control, affinity purified BACE1 was also incubated in the absence of the enzyme (HAT) and in the presence of pre-boiled (denatured) enzyme.

Purification of ER and Golgi Intact Vesicles

Intact vesicles from the endoplasmic reticulum (ER) and the Golgi apparatus were purified on a 10-34% Iodixanol (OptiPrep; Axis-Shield, Norway) continuous gradient as described (Puglielli et al., 2001, Nat. Cell Biol. 3: 905-912). The complete migration of subcellular markers in a typical gradient is shown in Costantini et al., 2006, EMBO J. 25: 1997-2006; Puglielli et al., 2001, Nat. Cell Biol. 3: 905-912. Fractions enriched in ER and Golgi markers (also called ER or Golgi vesicles herein) were separated, pooled together, and resuspended in isotonic/cryogenic buffer (0.25 M sucrose and 10 mM Tris-HCl, pH 7.4) in the presence of protease inhibitors (Roche). Latency of ER and Golgi vesicles was determined with the glucose-6-phosphatase (Clairmont et al., 1992, J. Biol. Chem. 267: 3983-3900) and the sialyl-transferase (Puglielli and Hirschberg, 1999, J. Biol. Chem. 274: 35596-35600) methods, respectively. The purity of the preparation was further confirmed by assaying the above enzymatic activities, together with transport of CMP-sialic acid. Approximately >95% of vesicles were sealed and of the same membrane topographical orientation compared to in vivo.

Trypsin Digestion of ER and Golgi Vesicles

Vesicles were incubated for 60 min at 25° C. with trypsin (Sigma, St. Louis, Mo.) at the final concentration of 1 μg of protease per μg of ER/Golgi proteins. Digestion was halted by the addition of anti-trypsin specific inhibitor (Sigma) and by lowering the temperature to 0-4° C. The anti-trypsin inhibitor was used at the final concentration of 1 μg of inhibitor per μg of protease. As control, 0.05% Triton X-100 was added in some experiments in order to allow access of trypsin to the lumen of ER and Golgi vesicles.

Acetyl-CoA Transport Assay

Transport of acetyl-CoA into ER and Golgi vesicles was performed as previously described for nucleotide sugars (Puglielli and Hirschberg, 1999, J. Biol. Chem. 274: 35596-35600; Puglielli et al., 1999, J. Biol. Chem. 274: 12665-12669) with some modifications. Briefly, assays were performed in 100 μl final volume (isotonic/cryogenic buffer) using 25 to 50 μg of ER/Golgi vesicles protein. The rate of acetyl-CoA uptake at different concentrations was determined maintaining the amount of radioactive acetyl-CoA constant, whereas unlabeled acetyl-CoA ranged between 0.125 and 100 μM. After 5 min at 30° C., reactions were stopped by lowering the temperature to 0-4° C. and by adding 100 μl of ice-cold isotonic/cryogenic buffer (described above). Reaction mixtures were immediately spun at 25 psi (100,000×g) for 15 min using an air-driven ultracentrifuge (Airfuge; Beckman, Fullerton, Calif.). Pellets were then dissolved in 0.6 mL of NaOH 1 N; after neutralization with 0.2 mL of 4N HCl, samples were counted by scintillation spectrometry. Transport of CMP-sialic acid and ATP was performed at two different concentrations (5 and 10 μM) of solute as described above and fully characterized in previous publications (Puglielli and Hirschberg, 1999, J. Biol. Chem. 274: 35596-35600; Puglielli et al., 1999, J. Biol. Chem. 274: 12665-12669).

Membrane Acetyltransferase/Deacetylase Activities

For the acetyltransferase activity from in vivo membranes, BACE1-myc was incubated with purified Golgi and ER membrane vesicles and [³H]Acetyl-CoA (1000 cpm/pmol) for 1 hour at 30° C. The reaction was performed in 200 μl of acetylation buffer (50 mM Tris HCl, pH 8.0), 0.1 mM EDTA, 1 mM DTT, 10% glycerol, 20 μM acetyl-CoA) in the presence or absence of 0.2% Triton X-100. The reaction was stopped by adding 200 μl of ice-cold buffer and immediate immersion in ice; BACE1 was then immunoprecipitated and counted on a liquid scintillation counter. As a control, affinity purified BACE1 was also incubated in the absence of ER vesicles (no enzyme) and in the presence of membranes that had been boiled prior to the assay.

For the deacetylase activity, BACE1-myc was first acetylated in vitro by using the recombinant HAT fragment of p300 and [³H]Acetyl-CoA (as described above) and then purified again with magnetic beads cross-linked to anti-BACE1 antibodies. BACE1 was eluted by lowering the pH to 2.0 and recovered by centrifuging at 3000 rpm for 2 min. The pH was immediately neutralized by adding 10 μl of neutralizing buffer (1 M Tris, pH 9.5) per 200 μl of elution buffer. Acetylated BACE1 was then incubated in the presence of Golgi and ER membrane vesicles for 1 hr at 30° C. The reaction was performed in 200 μl of acetylation buffer (without EDTA) in the presence or absence of 0.2% Triton X-100, and stopped as above. BACE1 was then immunoprecipitated and counted on a liquid scintillation counter. The deacetylation assay was performed in the presence of the same controls described above.

Protein Labeling, Pro-Peptide Maturation, and Cell-Surface Biotinylation

Confluent cells were starved for 1 hr in Met/Cys free medium, labeled with 100 μCi [³⁵S]-Met/Cys (Trans35S-Label, >1000 Ci/mmol; Pierce) in starvation medium/dish and chased for 8, 16, and 24 hours with DMEM supplemented with 5 mM cold Met/Cys. Cell extracts were pre-cleared with BioMag Protein A (Polysciences, Inc.). Beads were used at the concentration of 15 μl of the slurry per sample and separated with a magnetic holder. BACE1 was then immunoprecipitated with anti-BACE1 antibodies and recovered with BioMag Protein A as above. The immunoprecipitate-bead complexes were washed 3 times and boiled in sample buffer. Beads were then separated and the samples were analyzed by reducing SDS-PAGE (on a 4-12% Bis-Tris NuPAGE system) and autoradiography.

For the analysis of pro-BACE1 maturation, cells were labeled and chased for 10 min and 2 hours as above. Pro-BACE1 was immunoprecipitated from total cell lysates with an antibody specific for the pro-domain of BACE1 and then recovered with BioMag Protein A. Unbound BACE1 was further immunoprecipitated with anti-BACE1 antibodies. The immunoprecipitate-bead complexes were washed 3 times and boiled in sample buffer. Beads were then separated and the samples were analyzed by reducing SDS-PAGE and autoradiography.

For cell-surface biotinylation, cells were labeled as above and chased for 1.5 hours. Cells were then washed twice, resuspended in PBS containing EZ-Link Sulfo-NHS-LC-Biotin (Pierce) at the final concentration of 2 mg/ml, and incubated for 30 min at room temperature. Biotinylated proteins were separated from cell extracts by using ImmunoPure Immobilized Streptavidin (as suggested by the manufacturer; Pierce). Proteins were eluted and recovered as above; BACE1 was then immunopurified and analyzed by reducing SDS-PAGE and autoradiography.

Statistical Analysis

Results are always expressed as mean±S.D. of the indicated number of determinations. The data were analyzed by ANOVA and Student's t-test comparison, using GraphPad InStat3 software (GraphPad, San Diego, Calif.). Statistical significance was reached at p<0.05.

BACE1 is Acetylated in the Lumen of the ER and Deacetylated in the Lumen of the Golgi Apparatus

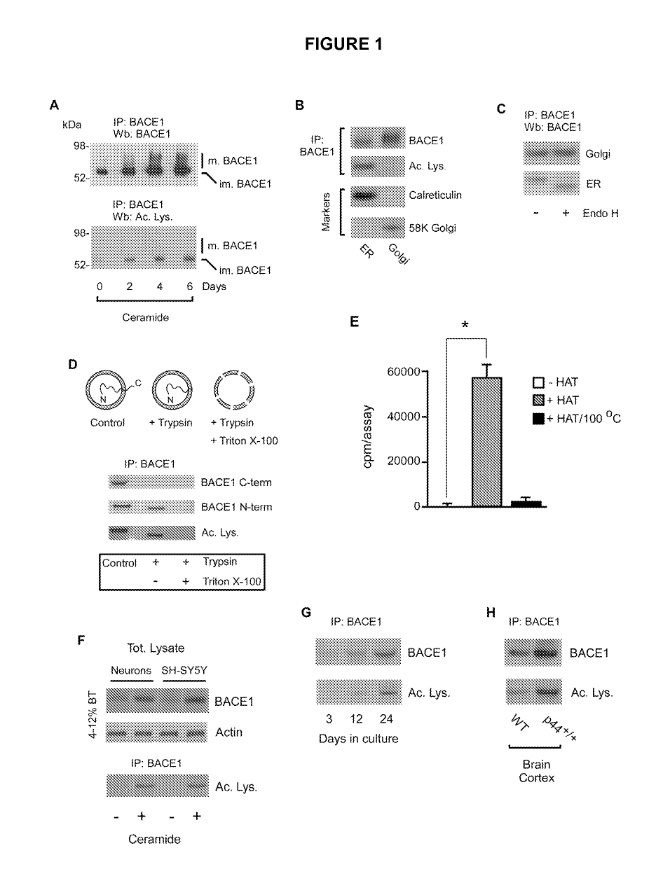

FIG. 1 illustrates how BACE1 is transiently acetylated in the lumen of the ER. For this experiment, CHO cells expressing human BACE1 were treated with C6-ceramide (also simply called ceramide herein) for different periods of time. BACE1 was then immunoprecipitated and analyzed by Western blotting. FIG. 1(A): CHO cells stably expressing BACE1 were treated with ceramide for the indicated time. BACE1 was immunoprecipitated from total cell lysates, separated on a 7% Tris-Acetate NuPAGE system, and then analyzed by Western blot using either anti-BACE1 or anti-acetylated lysine antibodies. Immature (im. BACE1) and mature (m. BACE1) BACE1 are indicated. FIG. 1(B): BACE1 was immunoprecipitated from ER and Golgi vesicles and then analyzed with the indicated antibodies. ER (calreticulin) and Golgi (58 Golgi protein) markers are shown. For this experiment, samples were separated on a 4-12% BisTris NuPAGE system, which allows a better resolution of total (mature and immature) BACE1. FIG. 1(C): BACE1 was immunoprecipitated from ER and Golgi vesicles as in (B) and then digested with Endoglycosidase H (Endo H) prior to immunoblotting. FIG. 1(D): intact ER vesicles were digested with trypsin for 30 minutes at 25° C., in the presence or absence of 0.05% Triton X-100. Digestion was halted using anti-trypsin specific inhibitor. BACE1 was then immunoprecipitated using a monoclonal antibody against the N-terminal domain and analyzed by Western blot using the indicated antibodies. FIG. 1(E): BACE1-myc was purified using an anti-myc affinity column and incubated in the presence of HAT and [³H]Acetyl-CoA for 1 hour at 30° C. BACE1 was then purified again and analyzed on a scintillation liquid counter. Asterisk (*) indicates statistical significance (n=4) (P<0.0005). FIG. 1(F): primary neurons and human neuroblastoma (SH-SY5Y) cell lines were treated with ceramide for 6 days. The steady-state levels of BACE1 (upper panel) were assessed by immunoblotting of total cell lysates, whereas the acetylation of BACE1 (lower panel) was assessed following BACE1 immunoprecipitation. Electrophoresis was performed on a 4-12% BisTris NuPAGE system. Neurons were prepared from wild-type mice as described in Costantini et al., 2005, Biochem. J. 391: 59-67; Costantini et al., 2006, EMBO J. 25: 1997-2006. FIG. 1(G): primary neurons were cultured in vitro up to 24 days as described (Costantini et al., 2005, Biochem. J. 391: 59-67; Costantini et al., 2006, EMBO J. 25: 1997-2006) and then analyzed for BACE1 acetylation with the indicated antibody. FIG. 1(H): BACE1 was immunoprecipitated from brain (cortex) extracts of 1 month old WT and p44^{+/+} mice (Costantini et al., 2006, EMBO J. 25: 1997-2006) and then analyzed for lysine acetylation with the appropriate antibody.

A progressive increase in the steady-state levels of both immature and mature (fully-glycosylated) BACE1 was observed, with an apparent plateau at day 4 (FIG. 1A). Surprisingly, the lower band migrating as the immature form of BACE1 could also be detected with an antibody raised against acetylated lysine residues suggesting that this form—and not the mature—is acetylated (FIG. 1A; lower panel). The pattern of acetylation would also suggest that the lysine modification occurs in the ER, where the immature form of the protein is synthesized and found. Therefore, ER and Golgi fractions were isolated and analyzed both the expression levels and acetylation status of BACE1. As expected, BACE1 was found to be enriched in the Golgi apparatus, when compared to the ER, and to migrate slightly higher than the ER-form on gel electrophoresis (FIG. 1B). However, analysis of immunoprecipitated BACE1 with an anti-acetylated lysine antibody revealed that BACE1 is acetylated in the ER compartment, but not in the Golgi apparatus (FIG. 1B). The sensitivity to Endoglycosidase H (Endo H) digestion of BACE1 purified from the ER (FIG. 1C) confirmed that it represents an immature and only partially glycosylated form. In fact, resistance to Endo H is acquired in the cis/medial Golgi apparatus where the “nascent” and incomplete oligosaccharide chain is processed and completed.

BACE1 is a type I membrane protein with one single transmembrane domain (˜17 residues) and a 24-amino acid long cytosolic tail. The large N-terminal domain with 460 amino acids faces the lumen of the ER and Golgi apparatus while being synthesized and translocated to the plasma membrane (PM). The primary sequence of BACE1 shows 16 lysine residues; however, only one is found in the cytosolic tail, being the very last residue of BACE1. In order to analyze whether BACE1 acetylation occurred on the endo-lumenal or cytosolic portion of the protein, intact ER vesicles were purified following ceramide treatment, and subjected them to trypsin digestion in the absence and presence of mild concentrations of Triton X-100. It is worth stressing that vesicles were sealed and of the same membrane topographical orientation as in vivo. In the absence of detergent, trypsin does not have access to the lumen of the vesicles and, therefore, can only digest the cytoplasmic tail of ER-membrane proteins (FIG. 1D). Analysis of intact ER vesicles prior to trypsin digestion allowed detection of BACE1 with antibodies directed against both the N- and C-terminal domains, and against acetylated lysine residues (FIG. 1D; control lane). Following proteolytic digestion in the absence of detergent, BACE1 was detected with antibodies against the N— but not the C-terminus, confirming successful removal of the cytosolic tail (FIG. 1D). This is further indicated by the slight change in protein migration on gel electrophoresis following trypsin treatment. Surprisingly, BACE1 could still be detected when antibodies against acetylated lysine residues were used, indicating that acetylation occurred in the lumen of the ER. Complete digestion of BACE1 in the presence of Triton X-100 served as further control confirming successful trypsin digestion (FIG. 1D). Both the Endo H sensitivity of the ER-form (FIG. 1C) and the inability of trypsin to digest the N-terminal domain of the protein in the absence of detergent (FIG. 1D) indicate that nascent BACE1 is correctly inserted in the ER membrane.

An in vitro system was used to assess whether BACE1 could serve as substrate for lysine acetylation. A myc-tagged (at the C-terminus) version of BACE1 (Puglielli et al., 2003, J. Biol. Chem. 278: 19777-19783) was purified from CHO cells grown in the absence of the second messenger ceramide. Purified BACE1 was then incubated in the presence of radiolabeled acetyl-CoA, as donor of the acetyl group, and the histone acetyl transferase (HAT) domain of recombinant CBP/p300, a well-characterized acetyl-CoA:lysine acetyltransferase localized in the cytosol. FIG. 1E shows that indeed BACE1 can be acetylated in vitro, provided that is incubated in the presence of an enzymatically active acetyltransferase.

The acetylation sites were mapped by comparing peptide masses (MS/MS) generated from tryptic digests of BACE1 purified from stably transfected cells grown in the absence or presence of ceramide (see Table 2). Analysis of mass spectra from several individual digests of BACE1 identified seven different lysine residues. Lys299 and Lys307 were found to be acetylated even in the absence of ceramide; all the other residues (Lys126, Lys275, Lys279, Lys285, and Lys300) were found to be acetylated only following ceramide treatment. No additional residue appeared to be acetylated or modified following fingerprinting analysis.

The pattern of acetylation of the region comprised between amino acid 286 and amino acid 317 of BACE1 resulted in the generation of several peptides with “overlapping” acetylated lysine residues (peak mass 807.5, 1548.1, 2788.3, and 1994.1). This phenomenon has already been described with histones (Wisniewski et al., 2007, Mol. Cell. Proteomics 6: 72-87) and is generally interpreted as caused by dynamic/transient acetylation of the lysine residues. Therefore, the above results represent additional evidence that the acetylation of BACE1 is a transient and dynamic event with different lysine residues being acetylated/deacetylated at the same time, while the protein undergoes post-translational maturation along the secretory pathway.

TABLE 2
List of identified tryptic peptides with acetylated lysine residues

| | | | |
|---|---|---|---|
| | | Mass | |
| Residue | Sequence with modification | measured | Condition |

| | | | | |
|---|---|---|---|---|
| K126 | KGVYVPYTQGK | (SEQ ID NO:26) | 1283.9 | Cer |
| K126 | YYQRQLSSTYRDLRKGVYVPYTQGK | (SEQ ID NO:27) | 3113.0 | Cer |
| K275 | REWYYEVIIVRVEINGQDLK | (SEQ ID NO:28) | 2567.7 | Cer |
| K275 + K279 | VEINGQDLKMDCK | (SEQ ID NO:29) | 1574.1 | Cer |
| K285 | EYNYTDK | (SEQ ID NO:30) | 974.7 | Cer |
| K307 | VFEAAVK | (SEQ ID NO:31) | 807.5 | Ctrl + Cer |
| K299 | SIVDSGTTNLRLPK | (SEQ ID NO:32) | 1548.1 | Ctrl + Cer |
| K299 + K307 | SIVDSGTTNLRLPKKVFEAAVKSIK | (SEQ ID NO:33) | 2788.3 | Ctrl + Cer |
| K300 + K307 | KVFEAAVKSIKAASSTEK | (SEQ ID NO:34) | 1994.1 | Cer |

As shown in Table 2, six of the acetylated peptides identified were mono acetylated, whereas three were di-acetylated. The acetylated lysine residues are shown in bold (K). In many cases the acetylation altered the digestion pattern, most likely by conferring resistance to trypsin. This is particularly evident for K126 in the 3113.0 peptide, K275 in the 1574.1 peptide, 299 in the 2788.3 peptide, and K307 in the 1994.1 peptide. In other cases, the acetylated lysine residue was found either at the beginning or at the end of the identified peptide, which is similar to reported acetylation patterns for histone H1 variants (Wisniewski et al., 2007, Mol. Cell. Proteomics 6: 72-87) and p53 (Sakaguchi et al., 1998, Genes & Dev. 12: 2831-2841).

One unusual situation was found with peptide 1994.1, which shows an uncleaved lysine residue (K310) flanked by isoleucine and alanine. Even though predicted to be cleaved, there was a peptide (amino acids 300-317) that corresponded to the sequence reported above. This situation is normally caused by posttranslational modification of the flanking residue; however, A311 did not appear to be modified in the analysis. Upon prolonged tryptic digestion, peak 1994.1 disappeared whereas the intensity of a peak mass at 1303.4, corresponding to the di-acetylated KVFEMVKSIK (SEQ ID NO:35), increased. Therefore, the above results were consistent with acetylation of K300 and K307 of peak 1994.1.

A prolonged tryptic digestion also resulted in the disappearance of peak 2788.3; this was accompanied by a parallel increase of peak mass 1548.1, corresponding to the mono-acetylated SIVDSGTTNLRLPK (SEQ ID NO:32) and 1261.4, corresponding to the mono-acetylated KVFEAAVKSIK (SEQ ID NO:36). The above results are consistent with acetylation of K299 and K307 of peak 2788.3. Since K300 also appeared to be acetylated (even though in a different peak mass, 1994.1), it is possible that a low-abundant tri-acetylated (K299, K300, and K307) peptide exists.

The above results indicate that BACE1 can be acetylated both in vivo and in vitro on seven lysine residues situated in the N-terminal and globular part of the protein, which faces the lumen of the ER. The ability of BACE1 to undergo lysine acetylation in vivo was further confirmed with primary neurons and human neuroblastoma (SH-SY5Y) cells on an endogenous BACE1 background (FIG. 1F). As previously demonstrated, primary neurons undergo a spontaneous activation of p75^{NTR} signaling, downstream of IGF1-R, which leads to a pronounced activation of endogenous ceramide and BACE1 levels (Costantini et al., 2006, EMBO J. 25: 1997-2006). Furthermore, p44^{+/+} transgenic animals, which display hyperactivation of IGF1-R signaling and an accelerated aging phenotype, have increased levels of ceramide in the brain, when compared to age-matched controls. In addition, the same animals have increased levels of BACE1 and increased production of amyloid β-peptide (Costantini et al., 2006, EMBO J. 25: 1997-2006). Therefore, the acetylation pattern of endogenous BACE1 was analyzed under those situations and in the absence of exogenous treatment. FIG. 1G shows that primary neurons in culture display a progressive increase in the levels of lysine acetylation of BACE1 that follows the same trend of ceramide activation described before (Costantini et al., 2006, EMBO J. 25: 1997-2006). In addition, 1 month old p44^{+/+} mice display increased acetylation of BACE1 in the cortex (FIG. 1H), further confirming that the post-translational event described here occurs in vivo, is not limited to transgenic BACE1, and does not require exogenous ceramide.

Not wanting to be bound by the following theory, these results also suggest that acetylation is a transient event, most likely requiring deacetylation in the Golgi apparatus following full maturation of the protein. Finally, the results indicate that ceramide treatment stimulates BACE1 acetylation by facilitating the efficiency of lysine acetylation; this could require changes either in the availability of the acetyl donor, the enzymatic activity of the acetyltransferase, or both.

Lysine Acetylation is Required for the Ceramide-Dependent Stabilization of BACE1

In order to analyze whether the lysine acetylation is required for the molecular stabilization of BACE1 induced by ceramide, the seven lysine residues described above were mutated to alanine. The Lys to Ala substitution precludes the acetylation of the protein and eliminates the negative charges introduced by the acetyl groups, therefore behaving as a “loss-of-function”. Next, the steady-state levels and acetylation of both the native (BACE1_{WT}) and the mutated (BACE1_{Ala}) form of BACE1 following ceramide treatment of stably transfected cells were analyzed.

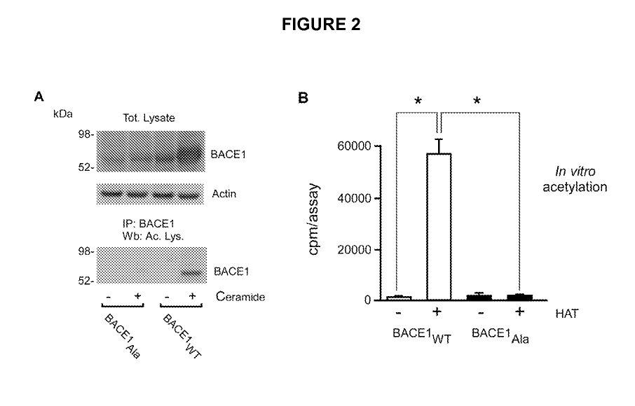

FIG. 2 shows how substitution of the lysine residues blocked the ability of BACE1 to be acetylated in vivo and in vitro. FIG. 2(A): CHO cells stably expressing the native (BACE1_{WT}) or mutated (BACE1_{Ala}) form of BACE1 were grown in the presence of ceramide and then analyzed by Western blot using an anti-BACE1 antibody (upper panel). Cell lysates were separated on a 4-12% BisTris SDS-PAGE system prior to Western blotting. For BACE1 acetylation, BACE1 was immunoprecipitated from total cell lysates and then analyzed using an anti-acetylated lysine antibody (lower panel). FIG. 2(B): both BACE1_{WT} and BACE1_{Ala} were purified from stably transfected cells using a specific anti-BACE1 antibody cross-linked to BioMag Protein A, eluted by lowering the pH, and then incubated in the presence of HAT and [³H]Acetyl-CoA for 1 hour at 30° C. BACE1 was then purified again and analyzed on a scintillation liquid counter. Asterisk (*) indicates statistical significance (n=3) (P<0.0005).

FIG. 2A (upper panel) shows that, in contrast to BACE1_{WT}, the steady-state levels of BACE1_{Ala} were not affected by ceramide treatment. In addition, ceramide did not induce acetylation of BACE1_{Ala}, indicating that this mutant form cannot be acetylated in vivo (FIG. 2A, lower panel). Incubation of purified BACE1 with the HAT domain of recombinant CBP/p300 in vitro resulted in acetylation of BACE1_{WT}, but not BACE1_{Ala}, further confirming that the mutated version of the protein cannot be acetylated and that the remaining nine lysine residues of the protein do not act as substrate for acetylation (FIG. 2B).

In contrast to alanine, glutamine is known to mimic the effect of lysine acetylation behaving as a bona-fide “gain-of-function”. Therefore, the seven lysine residues described above to glutamine (BACE1_{Gln}) were mutated, and the maturation process of both BACE1_{Ala} and BACE1_{Gln} were analyzed. For this purpose, investigation was conducted of how efficiently the pro-domain is removed from newly-synthesized BACE1. In fact, BACE1 contains a pro-peptide sequence at the N-terminus that is removed in the Golgi apparatus by a furin-like proprotein convertase (Bennett et al., 2000, J. Biol. Chem. 275: 37712-37717; Capell et al., 2000, J. Biol. Chem. 275: 30849-30854).

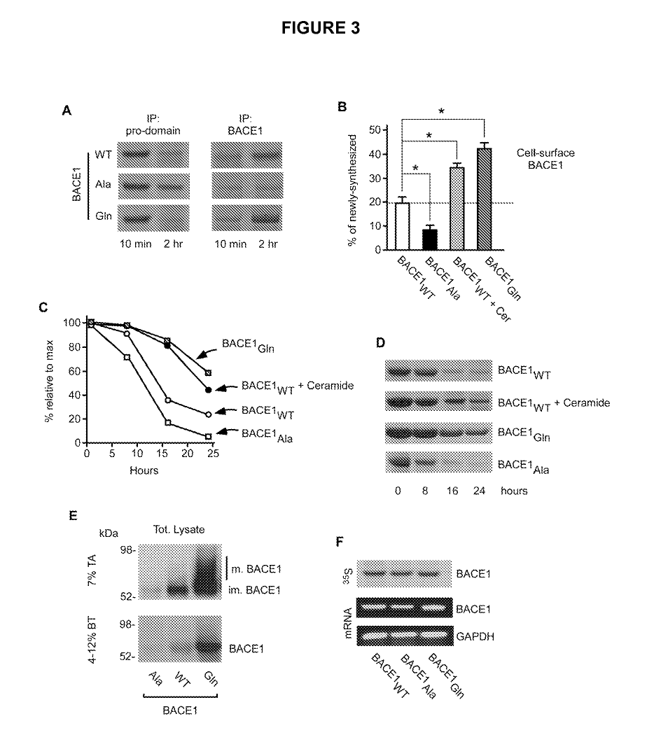

FIG. 3 illustrates how transient lysine acetylation controls both translocation along the secretory pathway and molecular stability of the nascent BACE1 protein. FIG. 3(A): BACE1_{WT}, BACE1_{Ala}, and BACE1_{Gln} cells were labeled with a mixture of radioactive Met/Cys for 10 min. and then chased for 2 hours. Pro-BACE1 was immunoprecipitated from total cell lysates with a specific antibody against the pro-domain region of BACE1. Unbound BACE1 (without the pro-domain) was then immunoprecipitated with an antibody against the C-terminal domain of BACE1. Immunoprecipitates were analyzed by SDS-PAGE and autoradiography. FIG. 3(B): BACE1_{WT}, BACE1_{Ala}, and BACE1_{Gln} cells were labeled with a mixture of radioactive Met/Cys for 10 min. and then chased for 1.5 hours. Cell surface proteins were biotinylated, separated with Immobilized Streptavidin, and eluted by lowering the pH. BACE1 was then immunoprecipitated as above and analyzed by reducing SDS-PAGE and autoradiography. Results are expressed as percentage of total newly-synthesized BACE1. Asterisks (*) indicate statistical significance (n=4) (P<0.005). FIG. 3(C and D): BACE1_{WT}, BACE1_{Ala}, and BACE1_{Gln} cells were labeled with a mixture of radioactive Met/Cys for 30 min. and then chased for 8, 16, and 24 hours. BACE1 was immunoprecipitated from total cell lysates and analyzed by SDS-PAGE and autoradiography. The half-life of BACE1 from five different readings is shown in FIG. 3(C), whereas a representative radiogram is shown in FIG. 3(D). FIG. 3(E): cell lysates from BACE1_{WT}, BACE1_{Ala}, and BACE1_{Gln} cells were separated either on a 7% Tris-Acetate or on a 4-12% Bis-Tris SDS-PAGE system, blotted onto a PVDF membrane, and probed with anti-BACE1 antibodies. Both immature (im. BACE1) and mature (m. BACE1) BACE1 are indicated. FIG. 3(F): Autoradiography of BACE1 immunoprecipitated from stably transfected cells immediately after a 30 min period of labeling with [³⁵S]Met/Cys (upper panel). Samples were separated on a 4-12% Bis-Tris SDS-PAGE system. BACE1 and GAPDH (glyceraldehyde-3-phosphate dehydrogenase; control lane) mRNA levels were quantified after total RNA extraction and RT-PCR (lower panel).

Pulse-chase experiments showed that BACE1_{Ala} displays a marked delay in the removal of the pro-domain sequence, when compared to either BACE1_{WT} or BACE1_{Gln} (FIG. 3A; left panel). This was also accompanied by reduced and delayed appearance of the cleaved BACE1 product (FIG. 3A; right panel). Finally, the removal of the pro-domain occurred faster and more efficiently with BACE1_{Gln} (FIG. 3A), suggesting a more efficient ER to Golgi translocation. This conclusion was further confirmed upon analysis of how efficiently the different mutant forms of BACE1 were able to reach the cell surface along the secretory compartment. Indeed, pulse-chase and cell-surface biotinylation experiments indicated that ˜9% of newly-synthesized BACE1_{Ala} is found at the cell-surface 1.5 hours after the pulse, compared to ˜20% of the native counterpart and to ˜42% of BACE1_{Gln} (FIG. 3B). It is worth noting that ceramide treatment of BACE1_{WT} expressing cells produced a similar effect by increasing the translocation of newly-synthesized BACE1 to the cell surface to levels almost similar to BACE1_{Gln} (FIG. 3B).

Ceramide treatment increases the half-life of both newly-synthesized and pre-formed BACE1. Therefore, the half-life of both the native and mutated forms of BACE1 following labeling with a radioactive mixture of methionine and cysteine was analyzed. The half-life of native BACE1 was ˜16 hours, but increased to ˜24 hours following ceramide treatment (FIGS. 3C and 3D). However, BACE1_{Ala} showed a significant reduction in the half-life with only ˜5% still detectable 24 hr after labeling, compared to ˜22% of BACE1_{WT} and ˜55% of BACE1_{Gln} (FIGS. 3C and 3D). Once again, BACE1_{WT} under ceramide treatment behaved very similarly to BACE1_{Gln}, further suggesting that the neutralization of the positive charges of the lysine residues provided by the acetylation (and mimicked by glutamine) affects the molecular stability and/or conformational maturation of BACE1. Consistent with the above results, analysis of total cell lysates on a 7% Tris-Acetate electrophoresis system revealed a marked decrease in the steady-state levels of BACE1_{Ala} (FIG. 3E). The overall reduction in the levels of the mutant protein was also evident when the same samples were analyzed on a 4-12% Bis-Tris gel, which resolves both mature and immature forms of BACE1 in one single band (FIG. 3E). In contrast to BACE1_{Ala}, BACE1_{Gln} showed a marked increase in the steady-state levels, when compared to BACE1_{WT}. These results were observed in the absence of any difference in the levels of BACE1 mRNA (FIG. 3F) or in the rate of incorporation of a radiolabeled methionine/cysteine mixture into the different BACE1 versions (FIG. 3F) excluding any defect in either transcription or translation induced by the mutagenesis.

Even though the results obtained with the lysine to alanine substitution were supported by those obtained with the lysine to glutamine substitution, the possibility exists that they were in part caused by the generation of an unstable protein that is not able to fold. Therefore, the same lysine residues were mutated to arginine (BACE1_{Arg}), which is thought to mimic more closely the lysine structure and to be a less perturbing mutation than alanine.

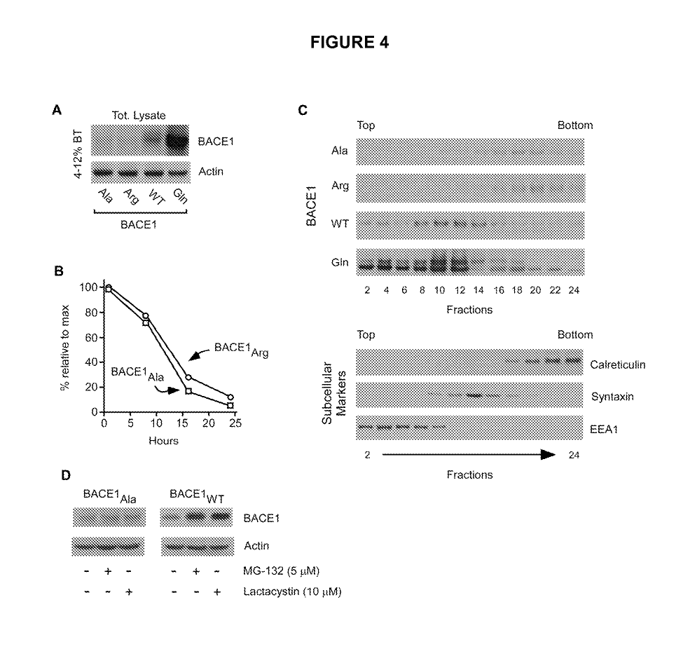

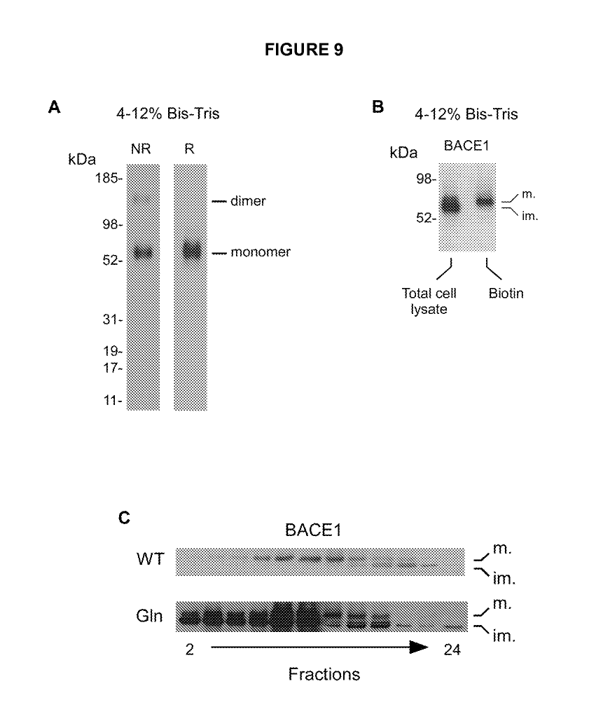

FIG. 4 illustrates how both the Lys to Ala and Lys to Arg (loss-of acetylation) substitutions generate an unstable BACE1 protein that is retained in the early secretory pathway and degraded by a proteasome-independent system. FIG. 4(A): the steady-state levels of BACE1_{Arg} were analyzed as described in FIG. 3E. FIG. 4(B): the half-life of BACE1_{Arg} was analyzed as in FIG. 3C and compared to BACE1_{Ala} (n=3). FIG. 4(C): the cellular distribution of the wild-type and mutant forms of BACE1 was analyzed by SDS-PAGE and immunoblotting after separation of intracellular membranes on a 10-24% discontinuous Nycodenz gradient. Membranes were developed in parallel on the same film. The appropriate subcellular markers are indicated: calreticulin (ER), syntaxin (Golgi apparatus), EEA1 (endosomes). A longer exposure of BACE1_{WT} and BACE1_{Gln} gradients showing the relative distribution of mature and immature BACE1 is found in FIG. 9C. FIG. 4(D): BACE1_{Ala} and BACE1_{WT} expressing CHO cells were treated with two different proteasome inhibitors for 10 hours. BACE1 steady-state levels were assessed by immunoblotting of total cell lysates on a 4-12% Bis-Tris NuPAGE system. The effects of proteasome inhibition on wild-type BACE1 have been described before (Qing et al., 2004, FASEB J. 18: 1571-1573).

Analysis of both the steady-state levels (FIG. 4A) and half-life (FIG. 4B) of BACE1 with either the Lys to Ala or the Lys to Arg substitutions did not seem to show any apparent difference, being both below the levels observed with BACE1_{WT}. Thus, the above results indicate that the neutralization of the positive charges of the lysine residues provided by the acetyl groups (and mimicked by the Lys to Gln substitution) is required for an efficient translocation of the nascent protein to the Golgi apparatus and to the cell surface along the secretory compartment. The retention of non-acetylated species of BACE1 in the early secretory pathway is further shown by the subcellular distribution pattern of the wild-type and mutated forms of BACE1 (FIG. 4C). In fact, the “loss-of-acetylation” mutants (BACE1_{Ala} and BACE1_{Arg}) were mostly observed in fractions corresponding to the ER and showed very low levels of mature BACE1. In contrast, the “gain-of-acetylation” mutant (BACE1_{Gln}) displayed a predominant localization in the late-Golgi and early-endosomal compartments with high levels of the mature form of the protein (visible as a double band in FIG. 4C; see also FIG. 9C). This distribution is consistent with previously reported localization of BACE1 and with the normal distribution of P secretase activity in the cell.

Even though the Lys to Ala substitution impaired the ability of the nascent BACE1 protein to leave the ER, there was no accumulation of the protein, suggesting a rapid and efficient removal of the non-acetylated intermediates (FIG. 3A). Inhibition of the proteasome machinery with either MG-132 or lactacystin successfully increased the steady-state levels of BACE1_{WT} but not those of BACE1_{Ala} (FIG. 4D) indicating that an alternative system degrades the non-acetylated intermediates of nascent BACE1.

Acetyltransferase and Deacetylase Activities Exist in the ER and Golgi Apparatus, Respectively

The above results indicate that the lysine acetylation of BACE1 is a transient event: the lysine residues are acetylated in the lumen of the ER and then deacetylated in the Golgi apparatus while the nascent protein moves ahead in the secretory pathway. This conclusion is supported by the fact that only the immature and ER-based form of BACE1 is acetylated (FIG. 1) and by the fact that the gain-of-acetylation mutant (BACE1_{Gln}) can move ahead in the secretory pathway, whereas the loss-of-acetylation (BACE1_{Ala} and BACE1_{Arg}) are retained in the ER (FIGS. 3 and 4).

However, for the above events to occur, the ER must possess an acetyl-CoA:lysine acetyltransferase, whereas the Golgi apparatus must possess a lysine deacetylase. In both cases, the catalytic site of the enzyme is predicted to face the lumen of the organelles. Since the donor of the acetyl group, acetyl-CoA, is highly charged and unable to cross the ER membrane, the results also predict a membrane transporter responsible for the translocation of acetyl-CoA from the cytoplasm to the lumen of the ER, where it can then serve as donor for the biochemical reaction.

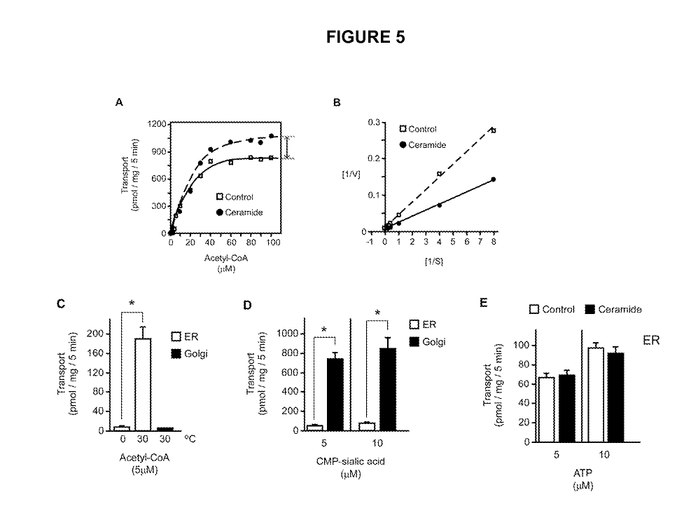

The above predictions were tested by analyzing each enzymatic step in vitro from highly purified intact ER and Golgi vesicles. Once again, vesicles were sealed and of the same membrane topographical orientation as in vivo. FIG. 5 illustrates how the ER membrane has an acetyl-CoA transport activity. FIG. 5(A and B): intact ER vesicles were incubated for 5 min at 30° C. with increasing concentrations of acetyl-CoA while maintaining [³H]Acetyl-CoA constant. The points of the double reciprocal plot in FIG. 5(B) illustrate a Lineweaver-Burk transformation of the data shown in A that were fitted by linear regression analysis to give a Kₘ of 14 μM and a Vₘₐₓ of 824 pmol/mg/5 min (control) (n=6). FIG. 5(C): ER and Golgi vesicles were assayed for transport of acetyl-CoA as described above. Asterisk (*) indicates statistical significance (n=3) (P<0.0005). FIG. 5(D): transport of CMP-sialic acid into ER and Golgi intact vesicles was measured as described in (A). The assay was performed at two different concentrations (5 and 10 μM) of solute. Asterisk (*) indicates statistical significance (n=3) (P<0.0005). FIG. 5(E): transport of ATP into ER intact vesicles obtained from control and ceramide-treated CHO cells was measured as described in FIG. 5(A). The assay was performed at two different concentrations (5 and 10 μM) of solute.

When ER and Golgi vesicles were incubated in the presence of increasing concentrations of acetyl-CoA, there was a progressive accumulation of acetyl-CoA in the lumen of ER-, but not Golgi-, derived vesicles (FIG. 5A-5C). Transport was saturable with an apparent Km of 14 μM and a Vmax of 824 μmol/5 min/mg of protein (FIGS. 5A and 5B). Finally, transport was temperature dependent (FIG. 5C), further suggesting that acetyl-CoA is translocated into the ER lumen in a carrier-mediated manner. Analysis of ER vesicles from ceramide treated cells revealed a ˜30% increase in the Vmax (1086±92 vs. 824±75 μmol/5 min/mg of protein; p<0.005), whereas the Km was comparable to untreated cells as demonstrated by the double reciprocal plot (Lineweaver-Burk transformation; FIG. 5B).

Analysis of CMP-sialic acid transport into ER and Golgi vesicles confirmed the purity of the in vitro system (FIG. 5D). Indeed, CMP-sialic transport only occurs in the Golgi apparatus, where it serves for sialylation of terminal galactose residues on both glycolipids and glycoproteins (Deutscher et al., 1984, Cell 39: 295-299). Finally, analysis of ATP transport into ER vesicles purified from control and ceramide-treated cells showed no apparent difference (FIG. 5E) indicating that the increased translocation of acetyl-CoA described in FIG. 5A is not caused by generalized changes in membrane permeability or transport activities of the ER following ceramide treatment.

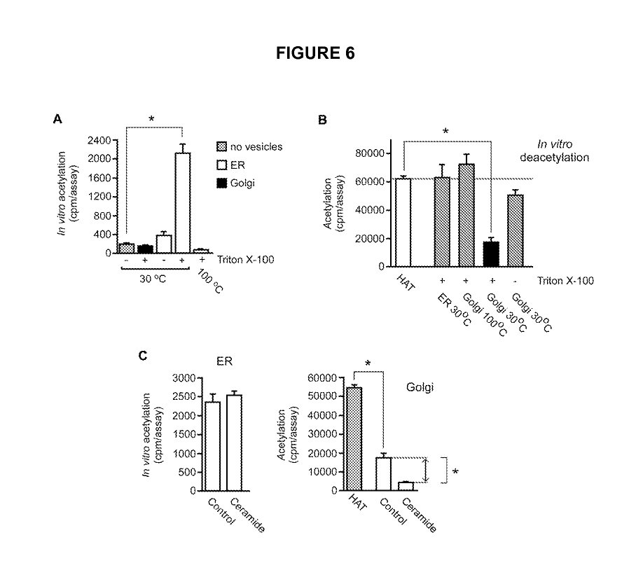

FIG. 6 illustrates how acetyltransferase and deacetylase activities exist in the lumen of ER and Golgi apparatus, respectively. FIG. 6(A): BACE1-myc was purified with an anti-myc affinity column, and then incubated with [³H]Acetyl-CoA and ER or Golgi vesicles in the presence/absence of 0.2% Triton X-100 for 1 hour at 30° C. The reaction was stopped by lowering the temperature; BACE1 was then purified again and analyzed on a scintillation liquid counter. As control, BACE1 was also incubated with [³H]Acetyl-CoA in the absence of ER/Golgi vesicles and with ER vesicles that had been boiled for 10 min prior to the reaction. Asterisk (*) indicates statistical significance (n=4) (P<0.0005). FIG. 6(B): purified BACE1-myc was first acetylated in vitro as described in FIG. 1E and then purified again in order to eliminate unbound acetyl-CoA. For the in vitro deacetylation, acetylated BACE1 was incubated with ER or Golgi intact vesicles in the presence/absence of 0.2% Triton X-100 for 1 hour at 30° C. Reaction was stopped by lowering the temperature; BACE1 was then immunoprecipitated and analyzed on a scintillation liquid counter. As control, BACE1 was also incubated with Golgi vesicles that had been boiled for 10 min prior to the reaction in order to inactivate any enzymatic activity. Asterisk (*) indicates statistical significance (n=4) (P<0.005). FIG. 6(C): both the in vitro acetylation and deacetylation of BACE1 were repeated by using intact ER (for acetylation) or Golgi (for deacetylation) vesicles prepared from control and ceramide-treated CHO cells. The assays were performed at 30° C. and in the presence of 0.2% Triton X-100, as described in panel A (for acetylation) or B (for deacetylation). Asterisks (*) indicate statistical significance (n=4) (P<0.0005).

Incubation of purified BACE1 with radiolabeled acetyl-CoA and ER vesicles revealed the existence of an acetyl-CoA:lysine acetyltransferase activity in ER vesicles that could not be observed in the absence of mild concentrations of Triton X-100, indicating that the catalytic site of the enzyme faces the lumen of the ER (FIG. 6A). In addition, the acetyltransferase activity was not detected in Golgi purified vesicles confirming that the enzyme is an ER resident protein (FIG. 6A).

BACE1 was incubated with radiolabeled acetyl-CoA and the HAT domain of CBP/p300. The in vitro acetylated BACE1 was then purified again and incubated with either ER or Golgi vesicles in the presence or absence of Triton X-100. Incubation with permeabilized Golgi vesicles resulted in a dramatic decrease in the levels of acetylation of BACE1 (FIG. 6B); such an effect was not observed when the incubation was done in the presence of ER or pre-boiled Golgi vesicles (FIG. 6B). Deacetylation of BACE1 was only observed when the Golgi vesicles were permeabilized with Triton X-100 (FIG. 6B) indicating that the active site of the deacetylase faces the lumen of the Golgi apparatus.

There was no apparent difference when the in vitro acetylation assay was performed using ER membrane vesicles purified from either control or ceramide-treated cells (FIG. 6C; left panel), suggesting that ceramide does not act directly on the acetyltransferase—at least in vitro. However, there was increased deacetylation of BACE1 when the in vitro deacetylation assay was performed with Golgi vesicles purified from ceramide-treated cells (FIG. 6C; right panel). The above results indicate that the ER possesses both an acetyl-CoA membrane transporter and an acetyltransferase activity, whereas the Golgi apparatus possesses a deacetylase activity. Finally, they also indicate that the efficiency of both acetyl-CoA translocation across the ER membrane and deacetylation in the Golgi apparatus is stimulated by the second messenger ceramide.

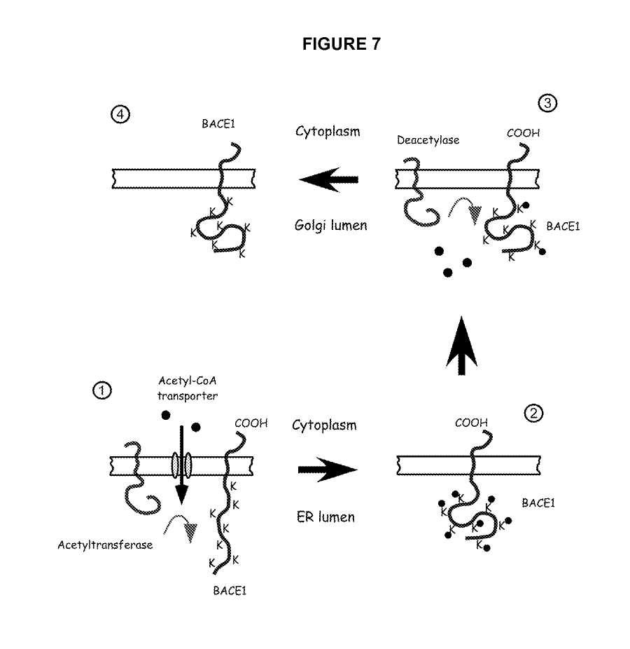

Not wanting to be bound by the following theory, a schematic model of a possible mechanism of BACE1 transient lysine acetylation/deacetylation is shown in FIG. 7. During translation/translocation across the ER membrane (1), BACE1 undergoes acetylation in seven different lysine residues facing the lumen of the organelle (ER). The reaction requires transfer of acetyl-CoA from the cytoplasm, where it is generated, to the lumen of the ER. The acetyl-CoA will then serve as donor of the acetyl group in the reaction of acetylation. This process is favored by the second messenger ceramide, thereby increasing the concentration of acetyl-CoA in the ER. The enzyme that carries out the reaction, the acetyl-CoA:lysine acetyltransferase, is also an ER resident protein with the catalytic site facing the lumen of the organelle. The acetylation of BACE1 provides conformational stability to the nascent protein (2). Once this has been achieved, BACE1 moves to the Golgi apparatus, where a Golgi-resident deacetylase will remove the acetyl groups (3). It is possible that this event is necessary in order to decrease the electron density of the globular domain of the protein (4) and allow conformational flexibility when the mature and active protein needs to shift from the ligand-free to the ligand-bound (and vice versa). In analogy with the many ER and Golgi resident glucosyltransferases already identified, both the acetyltransferase and the deacetylase are depicted here as membrane proteins.

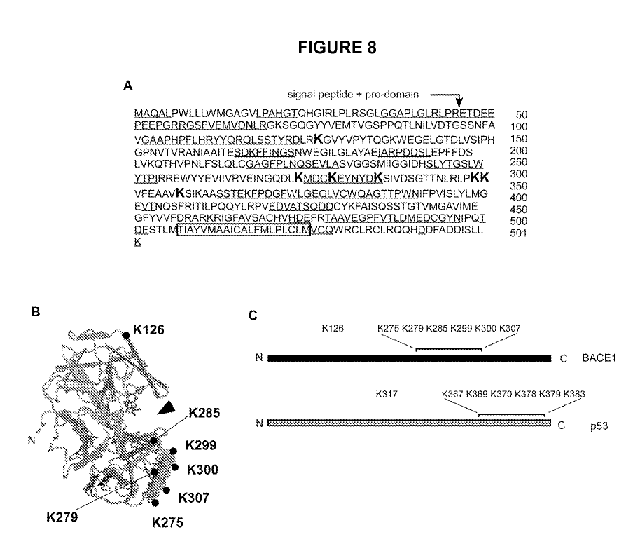

FIG. 8 illustrates how the acetylated lysine residues are clustered in a disordered region of BACE1. FIG. 8(A): disorder prediction of BACE1 protein sequence. Prediction was done by using DRIP-PRED analysis of the Stockholm Bioinformatics Center, Stockholm University, Sweden. Underlined regions scored >0.5 in the prediction algorithm and are probably disordered. The transmembrane domain is indicated by a box and appears highly ordered; the acetylated lysine residues are circled. FIG. 8(B): three-dimensional view of BACE1 with the acetylated lysine residues. The structure information, together with the graphic representation, was prepared using the Entrez's Molecular Modeling Database (MMDB) available from the National Center for Biotechnology Information (NCBI). The acetylated lysine residues are indicated by black circles. The single arrowhead indicates the catalytic site of the enzyme. FIG. 8(C): schematic view of the lysine residues that undergo acetylation in BACE1 and p53.

FIG. 9 shows images illustrating the non-reducing and reducing migration of BACE1. FIG. 9(A): BACE1 was immunopurified from total cell lysates and analyzed by Western blot under reducing (R) and non-reducing (NR) conditions. A high-molecular mass complex corresponding to dimeric BACE1 is visible only under non-reducing conditions. FIG. 9(B): cell surface BACE1 was biotinylated, separated with Immobilized Streptavidin, and eluted by lowering the pH. BACE1 was then immunoprecipitated and analyzed on a 4-12% BisTris NuPAGE system under reducing conditions. Biotinylated BACE1 migrates as mature BACE1. FIG. 9(C): subcellular distribution of BACE1_{WT} and BACE1_{Gln}. This figure represents a longer exposure of FIG. 4C.

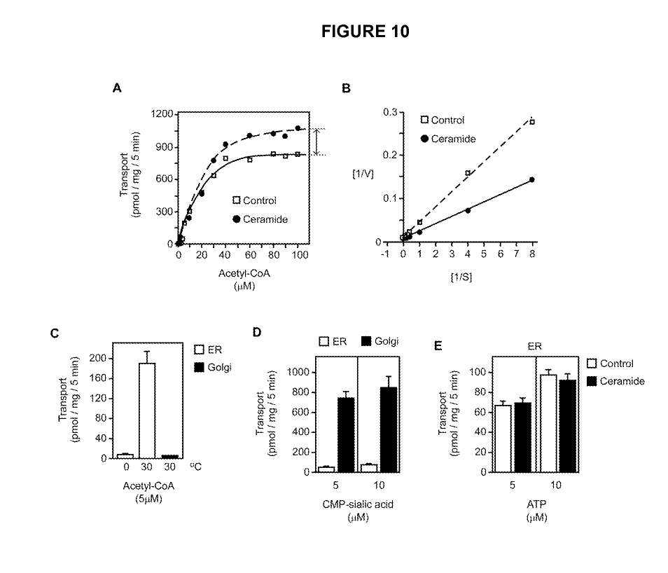

FIG. 10 shows images illustrating that the ER membrane has an acetyl-CoA transport activity. FIG. 10(A and B): intact ER vesicles were incubated for 5 min at 30° C. with increasing concentrations of acetyl-CoA while maintaining [³H]Acetyl-CoA constant. Translocation was measured as described in the Materials and Methods section. The points of the double reciprocal plot (Lineweaver-Burk transformation. FIG. 10(B): the data shown in A were fitted by linear regression analysis to give a Kₘ of 14 μM and a Vₘₐₓ of 824 pmol/mg/5 min (control). Results are the mean of at least three independent determinations. FIG. 10(C): ER and Golgi vesicles were assayed for transport of acetyl-CoA as described above. Results are the mean±SD of at least three independent determinations. FIG. 10(D): transport of CMP-sialic acid into ER and Golgi intact vesicles was measured as described in (A). The assay was performed at two different concentrations (5 and 10 μM) of solute. FIG. 10(E): transport of ATP into ER intact vesicles obtained from control and ceramide-treated CHO cells was measured as described in FIG. 10(A). The assay was performed at two different concentrations (5 and 10 μM) of solute.

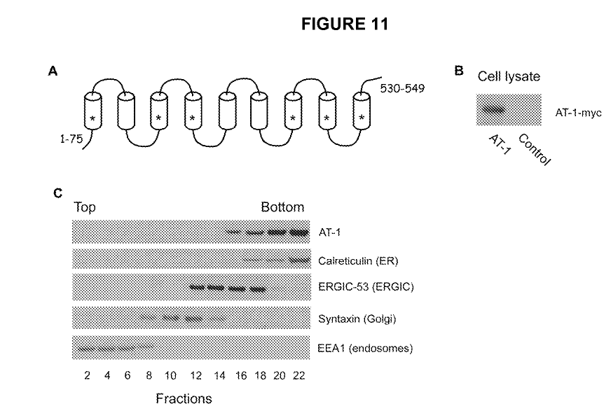

FIG. 11 illustrates how AT-1, an ER-based acetyl-CoA transporter migrates with ER markers. FIG. 11(A): schematic representation of AT-1. FIG. 11(B): expression of AT-1 in stable-transfected cells. FIG. 11(C): subcellular distribution of AT-1.

Identification and Biochemical Characterization of an ER-Based Acetyl-CoA Membrane Transporter

The data presented here indicate that the ER membrane has an acetyl-CoA transport activity that translocates acetyl-CoA from the cytoplasm to the lumen of the organelle. Since the acetyl-CoA serves as donor for the acetylation of nascent BACE1 in the lumen of the ER, the transport activity is required for the lysine acetylation machinery. An increased translocation of acetyl-CoA into the lumen of the ER, following ceramide treatment, results in stabilization of BACE1 and increased generation of AP. Reduced translocation of acetyl-CoA can impair the acetylation of nascent BACE1, resulting in lower steady-state levels of BACE1 and reduced generation of AP. This is supported by the fact that a defect in translocation of nucleotide-sugars (which act as donors of sugars in the reaction of glycosylation) into the Golgi apparatus results in a defect in the glycosylation of major glycoconjugates (Sturla et al., 2001, Pediatr. Res. 49: 537-541). Therefore, the identification and biochemical characterization of the membrane transporter is essential for the understanding of the molecular mechanisms that control the levels of BACE1, and may help us identify possible therapeutical strategies for the prevention of AD.

A putative acetyl-CoA transporter (named AT-1) was recently identified (Kanamori et al., 1997, Proc. Natl. Acad. Sci. USA 94: 2897-2902) and shown to have homologues in lower organisms, including S. cerevisiae, C. elegans, and D. melanogaster (Hirabayashi et al., 2004, Pflugers Arch. 447: 760-762). AT-1 has several features that suggest a role in the translocation of acetyl-CoA into the lumen of the ER (FIG. 11A), including (a) multiple membrane-spanning domains; (b) a leucine-zipper motif; and (c) an apparent ER localization. In addition, AT-1 is upregulated as result of ER-induced stress, suggesting a possible role during the unfolded protein response (Shaffer et al., 2004, Immunity 21: 81-93). However, until the present invention, there was no proof that AT-1 can act as an acetyl-CoA membrane transporter in vivo or in vitro.

Cellular Localization. CHO cells expressing human AT-1 with both a V5/His- and a myc-tag at the C-terminus were generated (FIG. 11B). The “tags” serve for the identification on Western blots and for the biochemical purification prior to the in vitro reconstitution. Biochemical fractionation studies indicate that AT-1 is localized in the ER and partially overlaps with the ERGIC (FIG. 11C). The V5/His-tag is used to confirm these results with immunofluorescence.

In Vivo Acetyl-CoA Transport Activity. ER intact vesicles were produced from both transfected and non-transfected cells, and then used to assay transport of acetyl-CoA into the lumen. If AT-1 acts as an ER-based acetyl-CoA membrane transporter, there is an increase in the rate of acetyl-CoA transport into ER vesicles, when compared to “control” ER. This system also allows to determine the biochemical characteristics (Kₘ and Vₘₐₓ), which are necessary to determine whether AT-1 corresponds to the transport activity that we have identified and described.

In Vitro Reconstitution. The reconstitution of a transport activity in an in vitro system is an approach to confirm the identity of a membrane transporter. For this purpose, AT-1 was initially purified by affinity chromatography (for example using a ProFound anti-myc column) and then reconstituted into a proteoliposome system as described before (Puglielli and Hirschberg, 1999, J. Biol. Chem. 274: 35596-35600; Puglielli et al., 1999, J. Biol. Chem. 274: 4474-4479; Puglielli et al., 1999, J. Biol. Chem. 274: 12665-12669). The reconstituted proteoliposomes serve to assay translocation of acetyl-CoA, as well as for the biochemical characterization. Analysis of Kₘ and Vₘₐₓ in vitro can serve to determine whether AT-1 corresponds to the transport activity that we have identified and described in native ER vesicles. In addition, analytical ultracentrifugation using an 8-30% glycerol gradient can be performed to determine the functional mass of AT-1.

The in vitro system can also be used to determine the recognition signals for AT-1. In the case of nucleotide-sugars, both the nucleotide and the sugar moieties are required for the transport activity. However, since they all act as antiporters, the nucleotide moiety is also a powerful inhibitor of the transport activity (Berninsone and Hirschberg, 2000, Curr. Opin. Struct. Biol. 10: 542-547). Therefore, the transport of acetyl-CoA can be assayed in the presence of increasing concentrations (10 to 100 μM) of the two individual moieties of the donor, the CoA and the acetyl group. These experiments can indicate the importance of the CoA moiety for recognition/activity. If the CoA moiety is critical for recognition/activity, biochemical compounds that mimic the structure of CoA may act as inhibitors of transport. This class of compounds could potentially serve to affect the levels of BACE1 and the rate of D cleavage of APP.

Endogenous/Tissue Expression Studies. Antibodies to endogenous AT-1 can be generated using methods known in the art, in order to confirm the subcellular localization and to analyze the tissue-specific expression of AT-1.

Depletion Assay. A depletion assay allows analyzing an enzymatic activity before and after elimination (depletion) of a specific enzyme/protein. In this case a protein extract of ER vesicles produced form control (non-transfected) cells is incubated with antibodies against endogenous AT-1 (similar to a classical immunoprecipitation assay). The immune complexes are then removed using protein A attached to magnetic bio-beads, and the resulting supernatant, which is depleted of AT-1, is reconstituted and assayed in vitro (as described above) for residual acetyl-CoA transport activity. The detergent contained in the supernatant is removed immediately before reconstitution using Extracti-Gel D columns, as described before (Puglielli and Hirschberg, 1999, J. Biol. Chem. 274: 35596-35600; Puglielli et al., 1999, J. Biol. Chem. 274: 4474-4479; Puglielli et al., 1999, J. Biol. Chem. 274: 12665-12669). This approach allows the determination of the contribution of AT-1 to the “endogenous” ER membrane acetyl-CoA transport activity. The detection of residual acetyl-CoA transport activity following biochemical depletion is suggestive of the presence of additional membrane transporters in the ER.

Down-Regulation of AT-1. Antisense oligonucleotides or siRNA probes can be designed, in order to down-regulate the expression levels of AT-1 in control cells. These experiments can allow determination of the effect of reduced acetyl-CoA transport into the ER lumen on the steady-state levels of BACE1 and on the rate of AP generation. The uses of both antisense oligonucleotides and siRNA have been described before (Costantini et al., 2006, EMBO J. 25: 1997-2006). In the case of antisense, the oligonucleotides are centered on the 5′ initiation codon and designed to maintain a GC:AT ratio of 60% in a frame of 11-15 nucleotides. Considering that the increased translocation of acetyl-CoA under ceramide treatment increased the steady-state levels of BACE1 and the production of AP, down-regulation of AT-1 can produce the opposite effect.

AT-1 Localizes in the ER and Stimulates Acetyl-CoA Transport Across the ER Membrane

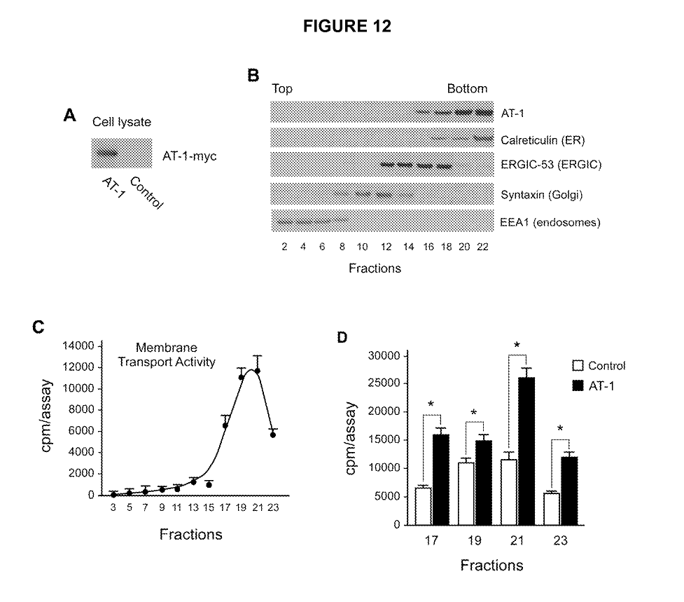

FIG. 12 shows images and graphs illustrating how AT-1 localizes in the ER and stimulates acetyl-CoA transport across the ER membrane. FIG. 12(A): Western blot analysis showing successful transfection of AT-1 into CHO cells. FIG. 12(B): the subcellular distribution of AT-1 was analyzed by SDS-PAGE and immunoblotting after separation of intracellular membranes on a 10-24% discontinuous Nycodenz gradient. The appropriate subcellular markers are indicated. FIG. 12(C): intracellular membranes from control CHO cells were prepared as in (B) and then assayed for acetyl-CoA transport activity. Results are the average±S.D. FIG. 12(D): the indicated fractions from control (non-transfected) and AT-1 stable transfected cells were assayed for acetyl-CoA transport. Results are the average±S.D. Asterisk (*) indicates statistical significance.

Acetyl-CoA Transport Across the ER membrane is Inhibited by Coenzyme A

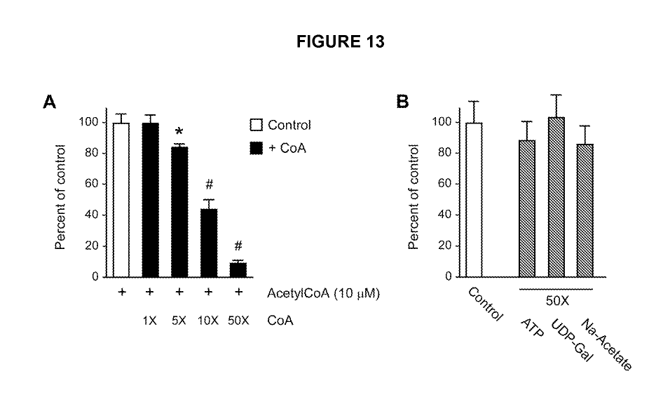

FIG. 13 shows graphs illustrating how acetyl-CoA transport across the ER membrane is inhibited by Coenzyme A. FIG. 13(A): ER vesicles were assayed for acetyl-CoA transport in the presence or absence of increasing concentrations of Coenzyme A (CoA). Results are expressed as percent of control (no CoA; white bar) and are the average±S.D. Asterisk (*) indicates statistical significance. FIG. 13(B): the experiment described in (A) was repeated in the presence of ATP, UDP-Galactose (UDP-Gal) or Na-Acetate.

AT-1 Regulates the Steady-State Levels of BACE1 and the Generation of Aβ

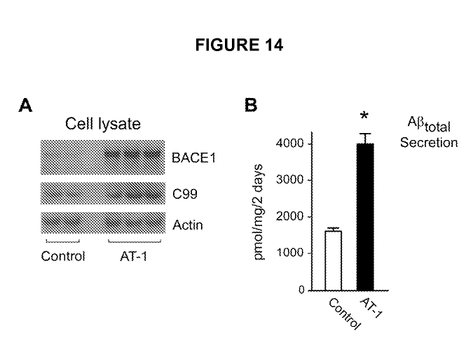

FIG. 14 shows an image and a graph illustrating how AT-1 regulates the steady-state levels of BACE1 and the generation of Aβ. FIG. 14(A): Western blot analysis showing the steady-state levels of BACE1 and C99 in control and AT-1 stable-transfected cells. FIG. 14(B): ELISA determination of total Aβ in the conditioned media of control and AT-1 stable-transfected cells.

The data presented above show that BACE1 undergoes transient acetylation on seven different lysine residues, all facing the N-terminal portion of the nascent protein. This process involves lysine acetylation in the lumen of the ER and is followed by deacetylation in the lumen of the Golgi apparatus. A dual-enzymatic machinery acts in the ER and Golgi apparatus respectively to acetylate and deacetylate the lysine residues (FIG. 7). In addition, this process requires carrier-mediated translocation of acetyl-CoA into the lumen of the ER and is stimulated by the lipid second messenger ceramide. Finally, transient/reversible lysine acetylation is required for nascent BACE1 to leave the ER and move ahead in the secretory pathway, and for the molecular stabilization of the protein.

Even though the native conformation of a protein lies encoded in its primary amino acid sequence, the efficiency of folding itself is greatly enhanced by the ER through different complex systems that require, among others, enzymes that modify, either temporally or definitively, the protein (Trombetta and Parodi, 2003, Annu. Rev. Cell Dev. Biol. 344, 649-676). An example of transient modification of a nascent protein that controls the efficiency of folding is the UGGT/calnexin system, which re-monoglucosylates nascent glycoproteins and regulates their interaction with the lectin chaperone calnexin (Kleizen and Braakman, 2004, Curr. Opin. Cell Biol. 16: 343-349). Once folding is completed, the protein is released from the calnexin cycle and left free to move ahead in the secretory pathway.

According to the data presented here, the acetylation/deacetylation system seems to work in the same way. Only immature BACE1 was found acetylated, thus suggesting that the process is transient. Indeed, the biochemical analysis identified both acetylase and deacetylase machineries that are physically separated: the acetylase machinery (acetyltransferase and acetyl-CoA transporter) was found only in the ER, whereas the deacetylase was found only in the Golgi apparatus. Substitution of all the lysine residues that can act as acceptors for the acetyl group into alanine generated a BACE1 protein (BACE1_{Ala}) that could not be acetylated in vivo or in vitro, and that was not affected by ceramide treatment. This mutated version of BACE1 showed a decreased efficiency in the translocation to the Golgi apparatus and to the plasma membrane, suggesting physical retention in the ER, and a reduced half-life. In contrast, when the same lysine residues were substituted with glutamine to mimic a constitutive acetylation, the new protein (BACE1_{Gln}) showed a longer half-life, together with increased efficiency in its translocation to the Golgi apparatus and the plasma membrane along the secretory pathway. The fact that the Lys to Ala substitution was also mimicked by the Lys to Arg substitution strengthens the results and seems to exclude possible artifacts produced by the mutagenesis. The results obtained with the Lys to Gln substitution mimicked very closely those obtained with wild-type BACE1 under ceramide treatment.

Disorder prediction of BACE1 revealed that five of the acetylated lysine residues were either in (Lys275, Lys279, Lys285) or close to (Lys126 and Ly307) weak electron density areas (FIG. 8A). Further comparison with the structural determinants and temperature factor (B-factors) distribution of BACE1 indicated that, with exception of Lys 126, all the acetylated lysine residues are clustered in a highly-disordered region of the globular part of the C lobe of the protein (FIG. 8B), which seems to be required for the conformational flexibility of the protein when shifting from the ligand-free to the ligand-bound (and vice versa) state.

Intrinsically unstructured protein domains are important for the activity of many proteins with very diverse functions, which include regulation of transcription and translation, signal transduction, and chaperone-assisted unfolding of kinetically-trapped folding intermediates (Dyson and Wright, 2005, Nat. Rev. Mol. Cell. Biol. 6: 197-208). The disordered region provides structure flexibility and allows complex molecular interactions that require movement, transition-state, or coupled folding and binding to a certain substrate. Incorrect folding of this region can ultimately interfere with the functional activity of the protein. Acetylation neutralizes the positive charges of the lysine residues, and can function as an electrostatic mechanism to help the globular domain of the protein to assume the correct folding state. Consistent with this prediction, long-time molecular dynamic simulation of both ligand-free and ligand-bound BACE1 indicates that the disordered regions of the globular domain of BACE1 allow for structural fluctuation of the enzyme, which is probably necessary for correct substrate binding.

The above assumptions seem supported by the post-translational marking of the histone N-terminal tails, also referred to as “histone code” (Berger, 2002, Curr. Opin. Genet. Dev. 12: 142-148). These are intrinsically disordered regions that are subject to post-translational modification by acetylation, methylation, and phosphorylation, and that are required to fluctuate in order to allow access to DNA. Another example is offered by the regulatory domain of p53, which shows intrinsically disordered regions that provide flexibility for the various conformational requirements of the protein. Similarly to BACE1, p53 has seven lysine residues that are acetylated (Scrable et al., 2005, Int. J. Biochem. Cell. Biol. 37: 913-919), six of which are concentrated in disorder regions of the protein (FIG. 8C). In contrast to p53, which is cytoplasmic, BACE1 is a membrane protein and the transitory acetylation occurs in the lumen of the ER and Golgi apparatus.

In addition to the acceptor substrate, the reaction of lysine acetylation requires a donor of the acetyl group -acetyl-CoA-, and an enzyme—acetyltransferase—that transfers the acetyl group from the donor to the acceptor. In order for the reaction to occur in the lumen of the ER, both the enzyme and the donor must be made available. The data of the present invention indicate that the ER membrane has an enzymatic activity that acts as acetyl-CoA:lysine acetyltransferase both in vivo and in vitro. The data also indicate that the active site of the acetyltransferase faces the lumen of the organelle, and that the ER membrane is able to translocate the donor (normally found in the cytoplasm) inside the ER. Both the transferase and the transporter were found to act only in the ER and not in the Golgi apparatus, thus providing biochemical confirmation of the model shown in FIG. 7. Conversely, an enzymatic activity able to function as a lysine deacetylase in vitro was identified in highly purified intact Golgi vesicles, and was shown to be absent in the ER.

Ceramide was shown to stimulate the efficiency of acetyl-CoA transport across the ER membrane. This fact alone could potentially explain the increased acetylation of BACE1 observed following ceramide treatment. Indeed, an increased concentration of acetyl-CoA in the lumen of the ER would favor the kinetic of the reaction (lysine acetylation) leading to more protein and/or more lysine residues being acetylated per unit of time. Only two lysine residues were found acetylated prior to ceramide treatment; this number increased to seven following ceramide treatment. These results appear consistent with the kinetics of acetyl-CoA translocation across the ER membrane. An increase in the concentration of the donor is expected to facilitate the reaction and allow the acetylation of all lysine residues. Obviously, this also seems to implicate that the kinetics of acetylation might be different for the different lysine residues, with the Lys299 and Lys307 occurring at lower concentrations of the donor. In addition to the transport of acetyl-CoA across the ER membrane, ceramide was also able to stimulate the rate of deacetylation in the lumen of the Golgi apparatus, therefore suggesting a common regulatory mechanism that controls the reversible/transient acetylation of ER/Golgi transiting membrane proteins.

Even though the Lys to Ala substitution impaired the ability of the nascent BACE1 protein to leave the ER, there was no accumulation of the protein intermediate. The steady-state levels of BACE1_{Ala} were not “normalized” by inhibiting the proteasomes, therefore suggesting that an alternative system degrades the pool of BACE1_{Ala} that is not able to complete maturation and translocation along the secretory compartment. For example, the disposal of non-acetylated BACE1_{Ala} could require a specific chaperone and/or protease. Indeed, the lysine acetylation might serve as a recognition or binding site for proteins that mediate structural organization or disposal of unfolded and kinetically-trapped folding intermediates.

AT-1 is Upregulated in AD Brains and Following Ceramide Treatment

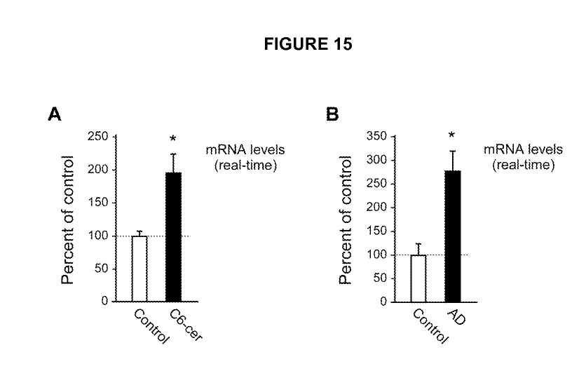

FIG. 15 shows graphs illustrating how AT-1 is upregulated in AD brains (brains of subjects affected with AD) and following ceramide treatment. FIG. 15(A): SH-SY5Y cells were treated with ceramide (C6-cer; 10 μM) prior to real-time quantitative PCR. Treatment was performed as previously described (Costantini et al., 2007; Costantini et al., 2005; Puglielli et al., 2003). Results are expressed as percent of control (n=5)±S.D. of control (no treatment). Asterisk (*) indicates statistical significance. No difference was observed in the mRNA levels of GAPDH, which was used as internal control. FIG. 15(B): a cDNA library (frontal cortex) of late-onset AD (n=5 different subjects) and age-matched controls (n=5 different subjects) was analyzed by quantitative real-time PCR. Results are expressed as percent of age-matched controls±S.D. Asterisk (*) indicates statistical significance. No difference was observed in the mRNA levels of GAPDH, which was used as internal control.

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters, obvious to those skilled in the art of biochemistry and molecular biology, are within the scope of this invention. All publications, patents, and patent applications cited herein are incorporated by reference in their entirety for all purposes.

What is claimed is:

1. A method, comprising:
a) reacting a protein, an ER-derived vesicle, and an acetyl-CoA; and
b) measuring the amount of post-translationally modified protein,
wherein the post-translational modification comprises acetylation of the protein in the ER-derived vesicle.

2. The method of claim 1, wherein the protein is aspartic peptidase.

3. The method of claim 2, wherein the aspartic peptidase is BACE1.

4. The method of claim 1, wherein the ER-derived vesicle comprises an acetyl-CoA transporter.

5. The method of claim 4, wherein the acetyl-CoA transporter is AT-1.

6. The method of claim 1, wherein providing a protein further comprises providing purified, isolated, or recombinant protein.

7. The method of claim 1, wherein the acetylation comprises acetylation of one or more lysine residues of the protein.

8. The method of claim 1 further comprising reacting the protein, the ER-derived vesicle, and the acetyl-CoA with a Golgi vesicle, wherein the post-translational modification comprises acetylation of the protein in the ER-derived vesicle, and deacetylation of the protein in the Golgi vesicle.

9. The method of claim 1, which is conducted in an animal model or clinical trials.

10. The method of claim 1, which is conducted in a cell culture.

11. A method for treating Alzheimer's disease, comprising reducing the acetyl-CoA transport activity of AT-1 in the endoplasmic reticulum of a subject with Alzheimer's disease.

12. The method of claim 11, comprising administering a therapeutically effective amount of an inhibitor of AT-1 activity to the subject, wherein the inhibitor of AT-1 activity reduces or eliminates the acetyl-CoA transport activity of AT-1 in the endoplasmic reticulum.

13. A method for treating Alzheimer's disease, comprising reducing the translocation of an aspartic peptidase from the ER into the Golgi of a subject with Alzheimer's disease.

14. The method of claim 13, wherein the aspartic peptidase is BACE1.

15. The method of claim 13, which is conducted in an animal model or clinical trials.

16. An in vitro method for identification of a candidate compound as a compound that may be useful for the treatment of Alzheimer's disease, the method comprising the steps of:
a) providing a cell expressing an enzyme that acetylates an aspartic peptidase in the ER, wherein the acetylation is required for translocation of the aspartic peptidase from the ER into the Golgi;
b) contacting the cell with the candidate compound; and
c) measuring the amount of aspartic peptidase translocated from the ER into the Golgi,
wherein a decrease in amount of the aspartic peptidase, relative to the amount of aspartic peptidase translocated from the ER into the Golgi by a cell expressing the enzyme but not contacted with the candidate compound, identifies the candidate compound as a compound that may be useful for the treatment of Alzheimer's disease.

17. The method of claim 16, wherein the aspartic peptidase is BACE1.

18. The method of claim 16, wherein the enzyme is AT-1.

19. An in vitro method for identification of a candidate compound as a compound that may be useful for the treatment of Alzheimer's disease, the method comprising the steps of:
a) providing a cell expressing an enzyme that acetylates BACE1 in the ER;
b) contacting the cell with the candidate compound; and
c) measuring translocation of the BACE1 from the ER into the Golgi,
wherein a decrease in the translocation, relative to the translocation by a cell expressing the enzyme but not contacted with the candidate compound, identifies the candidate compound as a compound that may be useful for the treatment of Alzheimer's disease.

20. The method of claim 19, wherein the enzyme is AT-1.

21. The method of claim 19, wherein the cell expresses a second enzyme that deacetylates BACE1 in the Golgi.

22. A screening method for testing a compound for its ability to inhibit acetylation of a protein in the ER, comprising:
a) reacting a protein that is acetylated in the ER, AT-1, and acetyl-CoA; and
b) measuring the amount of protein acetylated in the ER,
wherein a decrease in the amount of acetylated protein, relative to the amount of acetylated protein in the absence of the candidate compound, identifies the candidate compound as a compound that may be useful for the inhibition of protein acetylation in the ER.

23. The method of claim 22, wherein the protein is BACE1.

24. The method of claim 22, which is conducted in an animal model or clinical trials.

25. The method of claim 22, which is conducted in a cell culture.
